SEQUENCE LISTING (46 sequences)

SEQ ID NO: 1 - DNA, 3471 bp, Spinacia oleracea
  T10
atggctgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaattttatgtgaaagagatcctttgtaagattttagagatggttactaaggagaaagttgataatagttccgcattggaattggtacaaagccaatttcaagagaagttaagaggaaagaagtacttccttgttcttgatgatgtatggaacgaggatcgtgagaagtggtttcaattggaagagttgttaatgttgggtcaagggggaagcaaggttgtagtgaccgcacgttctgagaaaacagcaaatgtcatagggaaaagacatttatatacactggaatgtttgtcaccagattattcatggagcttatttgaaatgtcggcttttcagaaagggcaggagcaggaaaaccatcacgaactagttcgtattgggaaaaagattgttgaaaaatgttataacaatccacttgtgataaccgtggttggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttattacggagatgtttctttcaagatgtagtgaaggatgtatacggtgatgttaattctgttaaaatccacgacttgatgcacgatgtcgcccaagaagtggggagggaggaaatatgtgtagtgaatgataatacaaagaacttgggtgataaaatccgtcatgtacatggtgatgtcaatagatatgcacaaagagtctctctgtgtagccataagattcgttcgtatattggtggtgaatgtgaaaaaggttgggtggatacactaatagacaactggatgtgtcttagggtgttggacttgtcaaattcagatgttaaaagtttgcctaattcaataggtaagttgttgcacttacggtatcttaacctgtcaaataatagaaatctaaagatacttcctgatgcaattacaagactgcataatttgcagacactacttttatcttcgtgcagaagtttaaaggagttgccaaaagatttttgcaaattggtcaaactgagacacttggatttatggggttgttctgatttgattggtatgccattgggaatggataggctaactagtcttagagtactgccattctttgtggtgggtaggaaggaacaaagtgttgatgatgagctgaaagccctaaaaggcctcaccgagataaaaggctccatttctatcagaatctattcaaagtatagaatagttgaaggcatgaatgacacaggaggagctgcttatttgaagagcatgaaacatctcactggggttaatattatatttatatttgattataaaggtgtatttgttaaccctgaagctgtgttggaaaccctagagccaccttcaaatatcaagagcttatctatagataattacgatggtacaacaattccagtatggggaagagcaaagattaattgggcaatctccctctcacatcttgttgacatcaagcttgtaggttgtttatatttgcaggagatgccagtgctgagtaaactgcctcatttgaaatcactgtatcttgcgtggttgaataacttagagtacatggagagtagaagcagcagcagtagcagtgacacagaagcagcaacaccagaattaccaacattcttcccttcccttgaaaaacttaaactttggtatctggaaaagttgaagggtttggggaacaggagaccgagtagttttccccgcctctctaaattggtaatctgggaatgcccagatctaacgtggtttcctccttgtccaagccttgaaacgttgaaattggaaagaaacaatgaagcgttgcaaataatagtaaaaataacaacaacaagaggtaaagaagaaaaagaagaagacaagaatgctggtgttggaaattcacaagatgatgacaatgtcaaattatggaaggtgaaactagacaatgtgagttgtctcaaatcactgcccacaaattgtctgactcacctcgaccttacaataagtgattccaaggagggggagggtgaatgggaagttggggaggcatttcagaagtgtgtatcttctttgagaagcctcaccataatcggaaatcacggaataaataaagtgaagagactgtctggaagaacagggttggagcatttcactgccttacacttactcaaattttcagatatagaagaccaggaagatgagggcaaagacaacatcatattctggaaatcctttcctcaaaacctccgcagtttgataattcaatactcttataaaatgacaagtttgcccatggggatgcagtacttaacctccctccaaaccctcaaactagaaaattgtgatgaattgaattcccttccagaatggataagcagcttatcatctcttcaatccctgtacatatacaaatgtccagccctaaaatcactaccagaagcaatgcggaacctcacctcccttcagagacttgagatacagcattgtccagacctagctgaaagatgcagaaaacccaacggggaggactatcccaaaattcaacacatccccaaaattgtaagtcattgcagaaagtaa

SEQ ID NO: 2 - protein, 1156 aa, Spinacia oleracea
  T10
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNDNEACSFLTIVGAGGLGKTALAQLVFNDERVKIEFHDLRYWVCVSDQDGGQFYVKEILCKILEMVTKEKVDNSSALELVQSQFQEKLRGKKYFLVLDDVWNEDREKWFQLEELLMLGQGGSKVVVTARSEKTANVIGKRHLYTLECLSPDYSWSLFEMSAFQKGQEQENHHELVRIGKKIVEKCYNNPLVITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDVYGDVNSVKIHDLMHDVAQEVGREEICVVNDNTKNLGDKIRHVHGDVNRYAQRVSLCSHKIRSYIGGECEKGWVDTLIDNWMCLRVLDLSNSDVKSLPNSIGKLLHLRYLNLSNNRNLKILPDAITRLHNLQTLLLSSCRSLKELPKDFCKLVKLRHLDLWGCSDLIGMPLGMDRLTSLRVLPFFVVGRKEQSVDDELKALKGLTEIKGSISIRIYSKYRIVEGMNDTGGAAYLKSMKHLTGVNIIFIFDYKGVFVNPEAVLETLEPPSNIKSLSIDNYDGTTIPVWGRAKINWAISLSHLVDIKLVGCLYLQEMPVLSKLPHLKSLYLAWLNNLEYMESRSSSSSSDTEAATPELPTFFPSLEKLKLWYLEKLKGLGNRRPSSFPRLSKLVIWECPDLTWFPPCPSLETLKLERNNEALQIIVKITTTRGKEEKEEDKNAGVGNSQDDDNVKLWKVKLDNVSCLKSLPTNCLTHLDLTISDSKEGEGEWEVGEAFQKCVSSLRSLTIIGNHGINKVKRLSGRTGLEHFTALHLLKFSDIEDQEDEGKDNIIFWKSFPQNLRSLIIQYSYKMTSLPMGMQYLTSLQTLKLENCDELNSLPEWISSLSSLQSLYIYKCPALKSLPEAMRNLTSLQRLEIQHCPDLAERCRKPNGEDYPKIQHIPKIVSHCRK

SEQ ID NO: 3 - DNA, 3495 bp, Spinacia oleracea
  T70
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacatcgtgatgtcattagatatgcacaaagagtctctctgtgtagccataagattcgttcgtatattggtggtaaatgtgaaaaacgttgggtggatacactaatagacaagtggatgtgtcttaggatgttggacttgtcaaattcagatgttaaaagtttgcctaattcaataggtaagttgttgcacttacggtatcttaacctgtcaaataatagaaatctaaagatacttcctgatgcaattacaagactgcataacttgcagacactacttttagaagattgcagaagtttaaaggagttgccaaaagatttttgcaaattggtcaaactgaggcacttggatttaaggttttgttctgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgagggttattattagatttgatgataaagaaggtggatgtgttaaccctgaagctgtgttggcaaccctagagccaccttcaaatatcaagagcttatctatagataattacgatggtacaacaattccagtatggggaagagcagagattaattgggcaatctccctctcacatcttgtcgacatccagctttggcattgtcgtaatttgcaggagatgccagtgctgagtaaactgcctcatttgaaatcactgtatctttatactttgattagcttagagtacatggagagtagaagcagcagcagtagcagtgacacagaagcagcaacaccagaattaccaacattcttcccttcccttgaaaaacttacactttggtatctggaaaagttgaagggttttgggaacaggagaccgagtagttttccccgcctctctaaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatgtgagttatctcaaatcactgcccacaaattgtcttactcacctcaaaataactggaatagattacagggagggggagattgaatcagattccgtggaggaggagattgaattggaagttggggaggcatttcagaagtgtgcatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaacctccgcagtttggaaattgaaaactcttacaaaatgacaagtttgcccatggggatgcagtacttaacctccctccaaaccctctatctacaccatttttatgaattgaattcccttccagaatggataagcagcttatcatctcttcaatacctgcgcatatactactgtccagccctgaaatcactaccagaagcaatgcggaacctcacctcccttcagacacttgggatatcggattgtccagacctagttaaaagatgcagaaaacccaacggcaaggactatcccaaaattcaacacatccccaaaattgtaagtcattgcagaaagtaa

SEQ ID NO: 4 - protein, 1164 aa, Spinacia oleracea
  T70
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHRDVIRYAQRVSLCSHKIRSYIGGKCEKRWVDTLIDKWMCLRMLDLSNSDVKSLPNSIGKLLHLRYLNLSNNRNLKILPDAITRLHNLQTLLLEDCRSLKELPKDFCKLVKLRHLDLRFCSDLIGMPLGMDRLTSLRVLPFFVVGRKEQSVDDELKALKGLTEIKGSIRIRIHSKYRIVEGMNDTGGAGYLKSMKHLTRVIIRFDDKEGGCVNPEAVLATLEPPSNIKSLSIDNYDGTTIPVWGRAEINWAISLSHLVDIQLWHCRNLQEMPVLSKLPHLKSLYLYTLISLEYMESRSSSSSSDTEAATPELPTFFPSLEKLTLWYLEKLKGFGNRRPSSFPRLSKLEIWECPDLTWFPPCPSLKTLKLEKNNEALQIIVKITTTRGKEEKEEDKNAGVGNSQDDDNVKLRKVEIDNVSYLKSLPTNCLTHLKITGIDYREGEIESDSVEEEIELEVGEAFQKCASSLRSLIIIGNHGINKVMRLSGRTGLEHFTLLDSLKLSNIEDQEDEGEDNIIFWKSFPQNLRSLEIENSYKMTSLPMGMQYLTSLQTLYLHHFYELNSLPEWISSLSSLQYLRIYYCPALKSLPEAMRNLTSLQTLGISDCPDLVKRCRKPNGKDYPKIQHIPKIVSHCRK

SEQ ID NO: 5 - DNA, 3495 bp, Spinacia oleracea
  T71
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcattgataggttgcttaatcgtaatggtaatgaagcttgtagtttcctgaccatagtgggagcgggaggattgggaaaaactgctcttgcacaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccacattggaattggtgcaaagccaatttcaagagaagttaagaggaaagaagtacttccttgttctcgatgatgtatggaacgagggtcgtgagaagtggcttcatttggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataaataagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacatcgtgatgtcattagatatgcacaaagagtctctctgtgtagccataagattcgttcgtatattggtggtaaatgtgaaaaacgttgggtggatacactaatagacaagtggatgtgtcttaggatgttggacttgtcaaattcagatgttaaaagtttgcctaattcaataggtaagttgttgcacttacggtatcttaacctgtcaaataatagaaatctaaagatacttcctgatgcaattacaagactgcataacttgcagacactacttttagaagattgcagaagtttaaaggagttgccaaaagatttttgcaaattggtcaaactgaggcacttggatttaaggttttgttctgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgagggttattattagatttgatgataaagaaggtggatgtgttaaccctgaagctgtgttggcaaccctagagccaccttcaaatatcaagagcttatctatagataattacgatggtacaacaattccagtatggggaagagcagagattaattgggcaatctccctctcacatcttgtcgacatccagctttggcattgtcgtaatttgcaggagatgccagtgctgagtaaactgcctcatttgaaatcactgtatctttatactttgattagcttagagtacatggagagtagaagcagcagcagtagcagtgacacagaagcagcaacaccagaattaccaacattcttcccttcccttgaaaaacttacactttggtatctggaaaagttgaagggttttgggaacaggagaccgagtagttttccccgcctctctaaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatgtgagttatctcaaatcactgcccacaaattgtcttactcacctcaaaataactggaatagattacagggagggggagattgaatcagattccgtggaggaggagattgaattggaagttggggaggcatttcagaagtgtgcatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaacctccgcagtttggaaattgaaaactcttacaaaatgacaagtttgcccatggggatgcagtacttaacctccctccaaaccctctatctacaccatttttatgaattgaattcccttccagaatggataagcagcttatcatctcttcaatacctgcgcatatactactgtccagccctgaaatcactaccagaagcaatgcggaacctcacctcccttcagacacttgggatatcggattgtccagacctagttaaaagatgcagaaaacccaacggcaaggactatcccaaaattcaacacatccccaaaattgtaagtcattgcagaaagtaa

SEQ ID NO: 6 - protein, 1164 aa, Spinacia oleracea
  T71
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEGREKWLHLEELLMLGQGGSKVVVTARSEKTANVIGKRHFYTLECLSPDYSWSLFEMSAFQKGHEQENHDELVDIGKKIVEKCYNNPLAITVVGSLLYGEEINKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHRDVIRYAQRVSLCSHKIRSYIGGKCEKRWVDTLIDKWMCLRMLDLSNSDVKSLPNSIGKLLHLRYLNLSNNRNLKILPDAITRLHNLQTLLLEDCRSLKELPKDFCKLVKLRHLDLRFCSDLIGMPLGMDRLTSLRVLPFFVVGRKEQSVDDELKALKGLTEIKGSIRIRIHSKYRIVEGMNDTGGAGYLKSMKHLTRVIIRFDDKEGGCVNPEAVLATLEPPSNIKSLSIDNYDGTTIPVWGRAEINWAISLSHLVDIQLWHCRNLQEMPVLSKLPHLKSLYLYTLISLEYMESRSSSSSSDTEAATPELPTFFPSLEKLTLWYLEKLKGFGNRRPSSFPRLSKLEIWECPDLTWFPPCPSLKTLKLEKNNEALQIIVKITTTRGKEEKEEDKNAGVGNSQDDDNVKLRKVEIDNVSYLKSLPTNCLTHLKITGIDYREGEIESDSVEEEIELEVGEAFQKCASSLRSLIIIGNHGINKVMRLSGRTGLEHFTLLDSLKLSNIEDQEDEGEDNIIFWKSFPQNLRSLEIENSYKMTSLPMGMQYLTSLQTLYLHHFYELNSLPEWISSLSSLQYLRIYYCPALKSLPEAMRNLTSLQTLGISDCPDLVKRCRKPNGKDYPKIQHIPKIVSHCRK

SEQ ID NO: 7 - DNA, 3516 bp, Spinacia oleracea
  T72
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcattgataggttgcttaatcgtaatggtaatgaagcttgtagtttcctgaccatagtgggagcgggaggattgggaaaaactgctcttgcacaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccacattggaattggtgcaaagccaatttcaagagaagttaagaggaaagaagtacttccttgttctcgatgatgtatggaacgagggtcgtgagaagtggcttcatttggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataaataagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacatcgtgatgtcattagatatgcacaaagagtctctctgtgtagccataagattcgttcgtatattggtggtaaatgtgaaaaacgttgggtggatacactaatagacaagtggatgtgtcttaggatgttggacttgtcaaattcagatgttaaaagtttgcctaattcaataggtaagttgttgcacttacggtatcttaacctgtcaaataatagaaatctaaagatacttcctgatgcaattacaagactgcataacttgcagacactacttttagaagattgcagaagtttaaaggagttgccaaaagatttttgcaaattggtcaaactgaggcacttggatttaaggttttgttctgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgggggttgatattacatttgatggtggatgtgttaaccctgaagctgtgttggaaaccctagagccaccttcaaatatcaagagcttatctatagataattacgatggtacaacaattccagtatggggaagagcagagattaattgggcaatctccctctcacatcttgtcgacatcacgcttagttgttgtgaatatttgcaggagatgccagtgctgagtaaactgcctcatttgaaatcactgtatctttatactttgattagcttagagtacatggagagtagaagcagcagcagtagcagtgacacagaagcagcaacaccagaattaccaacattcttcccttcccttgaaaaacttacactttggtatctggaaaagttgaagggttttgggaacaggagaccgagtagttttccccgcctctctaaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatgtgagttatctcaaatcactgcccacaaattgtcttactcacctcaaaataactggaatagattacagggagggggagattgaatcagattccgtggaggaggagattgaattggaagttggggaggcatttcagaagtgtgcatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcattgtaagtgattgcagaaagtattttattcatttatatttattttatgcttag

SEQ ID NO: 8 - protein, 1171 aa, Spinacia oleracea
  T72
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEGREKWLHLEELLMLGQGGSKVVVTARSEKTANVIGKRHFYTLECLSPDYSWSLFEMSAFQKGHEQENHDELVDIGKKIVEKCYNNPLAITVVGSLLYGEEINKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHRDVIRYAQRVSLCSHKIRSYIGGKCEKRWVDTLIDKWMCLRMLDLSNSDVKSLPNSIGKLLHLRYLNLSNNRNLKILPDAITRLHNLQTLLLEDCRSLKELPKDFCKLVKLRHLDLRFCSDLIGMPLGMDRLTSLRVLPFFVVGRKEQSVDDELKALKGLTEIKGSIRIRIHSKYRIVEGMNDTGGAGYLKSMKHLTGVDITFDGGCVNPEAVLETLEPPSNIKSLSIDNYDGTTIPVWGRAEINWAISLSHLVDITLSCCEYLQEMPVLSKLPHLKSLYLYTLISLEYMESRSSSSSSDTEAATPELPTFFPSLEKLTLWYLEKLKGFGNRRPSSFPRLSKLEIWECPDLTWFPPCPSLKTLKLEKNNEALQIIVKITTTRGKEEKEEDKNAGVGNSQDDDNVKLRKVEIDNVSYLKSLPTNCLTHLKITGIDYREGEIESDSVEEEIELEVGEAFQKCASSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGISDCPDLVKICRKPNGEDYPKIQHIPGIVSDCRKYFIHLYLFYA

SEQ ID NO: 9 - DNA, 3531 bp, Spinacia oleracea
  T75
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcattgataggttgcttaatcgtaatggtaatgaagcttgtagtttcctgaccatagtgggagcgggaggattgggaaaaactgctcttgcacaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttcaattggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacatggtgatgtcaatagatatgcacaaagagtctctctgtgtagccataagattcgttcgtatattggtggtaattgtgaaaaacgttgggtggatacactaatagacaagtggatgtgtcttaggatgttggacttgtcaaggtcggatgttaaaaatttgcctaattcaataggtaagttgttgcacttaaggtatcttaacctgtcttataatgaagatctgttgatgctccctgatgcgattacaaaactgcataatttgcagactctacttttaaaatattgcagtggtttgaaggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagagcttatctatagataattacgatggtacaacaattccagtatggggaagagcagagattaattgggcaatctccctctcacatcttgtcgacatcacgcttagttgttatgaatatttgcaggagatgccagtgctgagtaaactgcctcatttgaaatcactgtatcttgttaggttgcataacttagagtacatggagagtagaagcagcagcagtagcagtgacacagaagcagcaacaccagaattaccaacattcttcccttcccttaaaacacttgtactttttgatctggaaaagttgaagggtttggggaacaggagaccgagtagttttccccgcctctctaaattggaaatcagggaatgcccagatctaacgtggtttcctccctgtccaagccttgaagagttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggtgataatagacaatgtgagttatctcaaatcactgcccacaaattgtcttactcacctcgacattacaataagagattccaaggagggggagggtgaatgggaagttggggaggcatttcagaagtgtgtatcttctttgagaagcctcatcataatcggaaatcacggaataaataaagtgaagagactgtctggaagaacagggttggagcatttcactctgttggactcactcaaactttcaaatatagaagaccaggaagatgagggcgaagacaacatcatattctggaaatcctttcctcaaaaccttcgcagtttgagaattgaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaaatgtccagccctaaaatcactaccagaagcaatgcggaacctcacctcccttcagagccttgtgatacggcggtgtccagacctgaaatcactaccagaagcaatgcggaacctcacctcccttcagagacttgagatacggcagtgtccagacctagctgaaagatgcagaaaacccaacggcgaggactatcccaaaattcaacacatccccaaaattgtaagtcattgcagaaagtaa

SEQ ID NO: 10 - protein, 1176 aa, Spinacia oleracea
  T75
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFQLEELLMLGQGGSKVVVTARSEKTANVIGKRHFYTLECLSPDYSWSLFEMSAFQKGHEQENHDELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHGDVNRYAQRVSLCSHKIRSYIGGNCEKRWVDTLIDKWMCLRMLDLSRSDVKNLPNSIGKLLHLRYLNLSYNEDLLMLPDAITKLHNLQTLLLKYCSGLKELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKSLSIDNYDGTTIPVWGRAEINWAISLSHLVDITLSCYEYLQEMPVLSKLPHLKSLYLVRLHNLEYMESRSSSSSSDTEAATPELPTFFPSLKTLVLFDLEKLKGLGNRRPSSFPRLSKLEIRECPDLTWFPPCPSLEELKLEKNNEALQIIVKITTTRGKEEKEEDKNAGVGNSQDDDNVKLRKVIIDNVSYLKSLPTNCLTHLDITIRDSKEGEGEWEVGEAFQKCVSSLRSLIIIGNHGINKVKRLSGRTGLEHFTLLDSLKLSNIEDQEDEGEDNIIFWKSFPQNLRSLRIEDSDKMTSLPMGMQYLTSLQTLELSYCDELNSLPEWISSLSSLQYLRIYKCPALKSLPEAMRNLTSLQSLVIRRCPDLKSLPEAMRNLTSLQRLEIRQCPDLAERCRKPNGEDYPKIQHIPKIVSHCRK

SEQ ID NO: 11 - DNA, 3465 bp, Spinacia oleracea
  T76
atggctgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgccgagtttatacctgtttgtagggaaagggggaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccgcacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgccgatattaaagctcagttaccataatcttataccctcgttgaagagttgcttcagttattgtgcagtgtttcccaaggatcatgaaataaagaaggagatgttgattgatctttggatagcacaaggatacgttgtggcacttgatggaggtcaaagtatagaagatgctgccgaagaacattttgtaattttgttacggagatgtttctttcaagatgtaaagaaggatgaatatggtgatgttgattctgttaaaatccacgacttgatgcacgatgtcgcccaagaagtggggagggaggaaatatgtgtagtgaatgataatacaaagaacttgggtgataaaatccgtcatgtacatcgtgatgtcattagatatgcacaaagagtctctctgtgtagccatagccataagattcgttcgtatattggtggtaattgtgaaaaacgttgtgtggatacactaatagacaagtggatgtgtcttaggatgttggacttgtcatggtcggatgttaaaaatttgcctaattcaataggtaaattgttgcacttgaggtgtcttaacctgtcttataataaagatctgttgatactccctgatgcaattacaagactgcataatttgcagacactgcttttaaaagattgcagaagtttaaaggagttgccaaaagatttttgcaaattggtcaaactgagacacttggatttatggggttgtgatgatttgattggtatgccatttggaatggataagctaactagtcttagaatactaccaaacattgtggtgggtaggaaggaacaaagtgttgatgatgagctgaaagcccttaaaggcctcaccgagataaaaggcgacattgatatcaaaatctgtgaaaattatagaatagttgaaggcatgaatgacacaggaggagctgggtatttgaagagcatgaaacatctcagggagattggtattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggactcactcaaattttcaaagatagaagaccaggaagatgagggcgaagacaacatcatattctggaaatcctttcctcaaaacctccgcagtttgagaattgaagactctgacaaaatgacaagtttgcccatggggatgcagtacttaacctccctccaaaccctctatctacaccattgttatgaattgaattcccttccagaatggataagcagcttatcatctcttcaatccctgcacatatacaaatgtccagccctaaaatcactaccagaagcaatgcggaacctcacctcccttcagagacttacgatatggcagtgtccagacctaattgaaagatgcaaagaacctaacggggaggactatcccaaaattcaacacatccccaaaattgtaagtcattgcagaaagtaa

SEQ ID NO: 12 - protein, 1154 aa, Spinacia oleracea
  T76
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTARSEKTANVIGKRHFYTLECLSPDYSWSLFEMSAFQKGHEQENHHELVDIGKKIVEKCYNNPLAITVVGSLLYGEEISKWRSFEMSELAKIGNGDNKILPILKLSYHNLIPSLKSCFSYCAVFPKDHEIKKEMLIDLWIAQGYVVALDGGQSIEDAAEEHFVILLRRCFFQDVKKDEYGDVDSVKIHDLMHDVAQEVGREEICVVNDNTKNLGDKIRHVHRDVIRYAQRVSLCSHSHKIRSYIGGNCEKRCVDTLIDKWMCLRMLDLSWSDVKNLPNSIGKLLHLRCLNLSYNKDLLILPDAITRLHNLQTLLLKDCRSLKELPKDFCKLVKLRHLDLWGCDDLIGMPFGMDKLTSLRILPNIVVGRKEQSVDDELKALKGLTEIKGDIDIKICENYRIVEGMNDTGGAGYLKSMKHLREIGITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLDSLKFSKIEDQEDEGEDNIIFWKSFPQNLRSLRIEDSDKMTSLPMGMQYLTSLQTLYLHHCYELNSLPEWISSLSSLQSLHIYKCPALKSLPEAMRNLTSLQRLTIWQCPDLIERCKEPNGEDYPKIQHIPKIVSHCRK

SEQ ID NO: 13 - DNA, 3489 bp, Spinacia oleracea
  T83
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcattgtaagtgattgcagaaagtattttattcatttatatttattttatgcttag

SEQ ID NO: 14 - protein, 1162 aa, Spinacia oleracea
  T83
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGISDCPDLVKICRKPNGEDYPKIQHIPGIVSDCRKYFIHLYLFYA

SEQ ID NO: 15 - DNA, 3495 bp, Spinacia oleracea
  T89
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaggatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataaataagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacatcgtgatgtcattagatatgcacaaagagtctctctgtgtagccataagattcgttcgtatattggtggtaaatgtgaaaaacgttgggtggatacactaatagacaagtggatgtgtcttaggatgttggacttgtcaaattcagatgttaaaagtttgcctaattcaataggtaagttgttgcacttacggtatcttaacctgtcaaataatagaaatctaaagatacttcctgatgcaattacaagactgcataacttgcagacactacttttagaagattgcagaagtttaaaggagttgccaaaagatttttgcaaattggtcaaactgaggcacttggatttaaggttttgttctgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgagggttattattagatttgatgataaagaaggtggatgtgttaaccctgaagctgtgttggcaaccctagagccaccttcaaatatcaagagcttatctatagataattacgatggtacaacaattccagtatggggaagagcagagattaattgggcaatctccctctcacatcttgtcgacatccagctttggcattgtcgtaatttgcaggagatgccagtgctgagtaaactgcctcatttgaaatcactgtatctttatactttgattagcttagagtacatggagagtagaagcagcagcagtagcagtgacacagaagcagcaacaccagaattaccaacattcttcccttcccttgaaaaacttacactttggtatctggaaaagttgaagggttttgggaacaggagaccgagtagttttccccgcctctctaaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatgtgagttatctcaaatcactgcccacaaattgtcttactcacctcaaaataactggaatagattacagggagggggagattgaatcagattccgtggaggaggagattgaattggaagttggggaggcatttcagaagtgtgcatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaacctccgcagtttggaaattgaaaactcttacaaaatgacaagtttgcccatggggatgcagtacttaacctccctccaaaccctctatctacaccatttttatgaattgaattcccttccagaatggataagcagcttatcatctcttcaatacctgcgcatatactactgtccagccctgaaatcactaccagaagcaatgcggaacctcacctcccttcagacacttgggatatcggattgtccagacctagttaaaagatgcagaaaacccaacggcaaggactatcccaaaattcaacacatccccaaaattgtaagtcattgcagaaagtaa

SEQ ID NO: 16 - protein, 1164 aa, Spinacia oleracea
  T89
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHDELVDIGKKIVEKCYNNPLAITVVGSLLYGEEINKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHRDVIRYAQRVSLCSHKIRSYIGGKCEKRWVDTLIDKWMCLRMLDLSNSDVKSLPNSIGKLLHLRYLNLSNNRNLKILPDAITRLHNLQTLLLEDCRSLKELPKDFCKLVKLRHLDLRFCSDLIGMPLGMDRLTSLRVLPFFVVGRKEQSVDDELKALKGLTEIKGSIRIRIHSKYRIVEGMNDTGGAGYLKSMKHLTRVIIRFDDKEGGCVNPEAVLATLEPPSNIKSLSIDNYDGTTIPVWGRAEINWAISLSHLVDIQLWHCRNLQEMPVLSKLPHLKSLYLYTLISLEYMESRSSSSSSDTEAATPELPTFFPSLEKLTLWYLEKLKGFGNRRPSSFPRLSKLEIWECPDLTWFPPCPSLKTLKLEKNNEALQIIVKITTTRGKEEKEEDKNAGVGNSQDDDNVKLRKVEIDNVSYLKSLPTNCLTHLKITGIDYREGEIESDSVEEEIELEVGEAFQKCASSLRSLIIIGNHGINKVMRLSGRTGLEHFTLLDSLKLSNIEDQEDEGEDNIIFWKSFPQNLRSLEIENSYKMTSLPMGMQYLTSLQTLYLHHFYELNSLPEWISSLSSLQYLRIYYCPALKSLPEAMRNLTSLQTLGISDCPDLVKRCRKPNGKDYPKIQHIPKIVSHCRK

SEQ ID NO: 17 - DNA, 250 bp, Artificial Sequence
  VIGS-construct
ggtgaatgtgttggccctgaagctgtattggaaaccttagagccaccttcaaatatcaagagcttatatatatataattacagtggtacaacaattccagtatggggaagagcagagattaattgggcaatctccctctcacatctcgtcgacatccagcttagttgttgtagtaatttgcaggagatgccagtgctgagtaaactgcctcatttgaaatcgctgaaacttggatggttggataacttag

SEQ ID NO: 18 - DNA, 400 bp, Artificial Sequence
  VIGS-construct
gagttcatgcgcttcaaggtgcgcatggagggctccgtgaacggccacagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttctcctccgacggccccgtaatgcagaagaagaccatgggctg

SEQ ID NO: 19 - DNA, 21 bp, Artificial Sequence
  primer sequence
ctcccctcaaccctaaagcaa

SEQ ID NO: 20 - DNA, 23 bp, Artificial Sequence
  primer sequence
gacaggacagcttggatgtttac

SEQ ID NO: 21 - DNA, 20 bp, Artificial Sequence
  Elongation factor Fwd
gcagggtcgttctttgagtc

SEQ ID NO: 22 - DNA, 20 bp, Artificial Sequence
  Elongation factor Rv
agaggctcttcctggtgaca

SEQ ID NO: 23 - DNA, 3447 bp, Spinacia oleracea
  T96
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcaattgtaagtga

SEQ ID NO: 24 - protein, 1148 aa, Spinacia oleracea
  T96
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGISDCPDLVKICRKPNGEDYPKIQHIPGNCK

SEQ ID NO: 25 - DNA, 3489 bp, Spinacia oleracea
  T253
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcattgtaagtgattgcagaaagtattttattcatttatatttattttatgcttag

SEQ ID NO: 26 - protein, 1162 aa, Spinacia oleracea; identical to SEQ ID NO: 14
  T253

SEQ ID NO: 27 - DNA, 3459 bp, Spinacia oleracea
  T18
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggaatcactcaaactttcagatatagaagaccaggaagatgagggcgaagacaacatcatattctggaaatcctttcctcaaaacctccgcagtttgagaattaaagactctgacaaaatgacaagtttgcccatggggatgcagtacttaacctccctccaaaccctctatctacaccattgttatgaattgaattcccttccagaatggataagcagcttatcatctcttcaatacctgcgcatatactactgtccagccctgaaatcactaccagaagcaatgcggaacctcacctcccttcagacacttgggatatcggattgtccagacctagttaaaagatgcagaaaacccaacggcaaggactatcccaaaattcaacacatccccaaaattgtaagtcattacagaaagtaa

SEQ ID NO: 28 - protein, 1152 aa, Spinacia oleracea
  T18
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLESLKLSDIEDQEDEGEDNIIFWKSFPQNLRSLRIKDSDKMTSLPMGMQYLTSLQTLYLHHCYELNSLPEWISSLSSLQYLRIYYCPALKSLPEAMRNLTSLQTLGISDCPDLVKRCRKPNGKDYPKIQHIPKIVSHYRK

SEQ ID NO: 29 - DNA, 3489 bp, Spinacia oleracea
  T133
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcattgtaagtgattgcagaaagtattttattcatttatatttattttatgcttag

SEQ ID NO: 30 - protein, 1162 aa, Spinacia oleracea
  T133
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGISDCPDLVKICRKPNGEDYPKIQHIPGIVSDCRKYFIHLYLFYA

SEQ ID NO: 31 - DNA, 3402 bp, Spinacia oleracea
  T139
atggccgaaatcggatactcggtttgtgcgaaactcatcgaagtgattggcagtgagctgatcaaagagatttgtgacacatggggttacaaatctcttcttgaggacctcaacaaaactgtattgacggtcaggaacgttctcattcaagccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaaggggaaacgagagggaaacacgttcatatatagatgtcaagaatattcttgggagggataaagataagaatgatatcatagataggttgcttaatcgtaatggtaatgaagcttgtagtttcctgaccatagtgggagcgggaggattgggaaaaactgctcttgcacaacttgtgttcaatgatgaaagggtcaaaattgagttccatgatttgaggtattgggtttgtgtctctgatcaagatgggggccaatttgatgtgaaagaaatcctttgtaagattttagaggtggttactaaggagaaagttgataatagttccacattggaattggtacaaagccaatttcaagagaagttaagaggaaagaagtacttccttgttcttgatgatgtatggaacgaagatcgtgagaagtggcttcctttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatcacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggaatcactcaaactttcagatatagaagaccaggaagatgagggcgaagacaacatcatattctggaaatcctttcctcaaaacctccgcagtttgagaattaaagactctgacaaaatgacaagtttgcccatggggatgcagtacttaacctccctccaaaccctctatctacaccattgttatgaattgaattcccttccagaatggataagcagcttatcatctcttcaatacctgcgcatatactactgtccagccctgaaaatcactaccagaagcaatgcggaacctcacctcccttcagagacttacggatatcggcattgtccagacctagttaaaagatgcagaaaaccctaa

SEQ ID NO: 32 - protein, 1133 aa, Spinacia oleracea
  T139
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNGNEACSFLTIVGAGGLGKTALAQLVFNDERVKIEFHDLRYWVCVSDQDGGQFDVKEILCKILEVVTKEKVDNSSTLELVQSQFQEKLRGKKYFLVLDDVWNEDREKWLPLEELLMLGQGGSKVVVTTRSEKTANVIGKRHFYTLECLSPDYSWSLFEMSAFQKGHEQENHHELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLESLKLSDIEDQEDEGEDNIIFWKSFPQNLRSLRIKDSDKMTSLPMGMQYLTSLQTLYLHHCYELNSLPEWISSLSSLQYLRIYYCPALKITTRSNAEPHLPSETYGYRHCPDLVKRCRKP

SEQ ID NO: 33 - DNA, 3489 bp, Spinacia oleracea
  T170
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgacgtatggaacgaggatcgtgagaagtggcttcgtttggaagagttgttaatgttgggtcaagggggaagcaaggttgtagtgaccacacgttcagagaagacagcaaatgtcatagggaaaagacatttttatacactggaatgtttgtcaccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgccgatattaaagctcagttaccataatcttataacctcgttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgataatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggatatcggattgtccagacctagttaaaatatgcagaaaacccaacggcgaggactatcccaaaattcaacacatccccggcattgtaagtgattgcagaaagtattttattcatttatatttattttatgcttag

SEQ ID NO: 34 - protein, 1162 aa, Spinacia oleracea
  T170
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRGRGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWLRLEELLMLGQGGSKVVVTTRSEKTANVIGKRHFYTLECLSPDYSWSLFEMSAFQKGHEQENHDELVDIGKKIVEKCYNNPLAITVVGSLLYGEEISKWRSFEMSELAKIGNGDNKILPILKLSYHNLITSLKSCFSYCAVFPKDHKIEKEMLIDLWIAQGYVVPLDGGQSIEDAAEEHFVILLRRCFFQDVVKDEYGDVDSVKIHDLMHDVAQEVGREEICIVNDNTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGISDCPDLVKICRKPNGEDYPKIQHIPGIVSDCRKYFIHLYLFYA

SEQ ID NO: 35 - DNA, 3363 bp, Spinacia oleracea
  T175
atggctgaaatcggatactcggtttgttcaaaacttattgaagtgatgggcagtaagatcattaaagagatttgcgacatgtggggttacaaaactcatcttgaagacctcaacaaatctgtcttgacaatcaaaaatgtgctcatggatgccgaggtgaagcgggatctttcccgtgtacaacagggttacattgcagaacttaaggatgttgtttacgatgctgatgatttgttcgatgagttcctcactcttgctgagctcaaacagattgatggcaactacaagggtggtggtaaattctctgaaaaggtacgtcgtttcttttcttctaataaggagaagatggatcaagcttacgacatgtctcgtaaggttaagaaaattaagaagcagttggatgaaattgttgataggcatacaaaatttgggtttattgttgattataaacctattattaggagaagggaggaaacatgttcttacgtagatgccaaggagattatcgggagagataaggataaggatgctatcattgatatgttgctagatcgtaatgataaggagggttgtagttttctgaccattgtgggggttggagggttggggaaaactgctcttgcccaacttgtgtataatgatgaaaaggtcataaaagagttcgagggtttgaggtattgggcttgtgtctctgatcaagacggggaggaatttgatgtgaaagcaatcctttgtaagattctagaatcagttactaaggtgaaacctgatggtagttccggattggaattggtgcaaaaccaatttcaggagagattaaggggaaagaagtacctccttgttcttgatgatgtatggaatgaggaccgtgagaagtggctttctttgaaaaagttcttaatgttaggtcaaaggggaagcaggattatggtaaccactcgttctaagacgacgacaaccatcataggggataaacatgcctatgaattacaaggtttatcccaagaggattcatggcacttgtttgagatttctgcatttgacaatgaatgtatccgccataatgagttcgttgagattggggagaagattgtttcgaaatgttatagcattcctcttgctataaaggtggtaggaagtcttctatttggccaggagatagttaagtggcagtcatttgaagcgagtggattgtcccaaattggcaatggtggtaatcaaatcatgtcaatattaaagctcagttatcacaatcttgcacactccttgaagagttgctttagttattgtgcagtattccccaaggatcataaaatagagaaagagatgttgattgacctttggatagcacaaggatatgttgtgccgttggatggtggtcaaagtatagaagatgctgccgaggaacattttgtaattttattacggagatgtttctttcaagatgtagtgaaggatgtatacggtgatgttaattctgttaaaatccacgacttgatgcacgacgtcgctcaagaagtagggagagaggaaatctgtatagtgaatgctaatacaaagaacttgggtgataaaatccgtcatgtacattgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtatcttaacctgtcaaataatagaaatctaaagatacttcctgatgcaattacaagactgcataacttgcagacactacttttagaagattgcagaagtttaatggagttgccaaaagatttttgcaaattggtcaaactgagacacttggatttatggggttgtgatgatttgattggtatgccattgggaatggatatgctaactagtcttagagtactgccatactttgtggtgggtaggaagaaacaaagtgttgatgatgagctgaaagccctaaaaggcctcaccgagataaaaggcgacattcgtattagaatctgttccaattatagaatagttgaaggcatgaatgacacaggaggagctgggtatttaaagagcatgaaacatctcacgggggttgatattacatttgatggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgcccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcatcataatcggaaatcacggaataaataaagtgatgagactgtctggaagaacagggttggagcatttcactctgttggactcactcaaactttcaaatatagaagaccaggaagatgagggcgaagacaacatcatattctggaaatcctttcctcaaaaccttcgcagtttgagaattaaagactctgacaaaatgacaagtttgcccatggggatgcagtacttaacctccctccaaaccctcgaactatcatattgtgatgaattgaattcccttccagaatggataagcagcttatcatctcttcaatacctgcgcatatacaactgtccagccctgaaatcactaccagaagcaatgcggaacctcacctcccttcagacacttgggggataa

SEQ ID NO: 36 - protein, 1120 aa, Spinacia oleracea
  T175
MAEIGYSVCSKLIEVMGSKIIKEICDMWGYKTHLEDLNKSVLTIKNVLMDAEVKRDLSRVQQGYIAELKDVVYDADDLFDEFLTLAELKQIDGNYKGGGKFSEKVRRFFSSNKEKMDQAYDMSRKVKKIKKQLDEIVDRHTKFGFIVDYKPIIRRREETCSYVDAKEIIGRDKDKDAIIDMLLDRNDKEGCSFLTIVGVGGLGKTALAQLVYNDEKVIKEFEGLRYWACVSDQDGEEFDVKAILCKILESVTKVKPDGSSGLELVQNQFQERLRGKKYLLVLDDVWNEDREKWLSLKKFLMLGQRGSRIMVTTRSKTTTTIIGDKHAYELQGLSQEDSWHLFEISAFDNECIRHNEFVEIGEKIVSKCYSIPLAIKVVGSLLFGQEIVKWQSFEASGLSQIGNGGNQIMSILKLSYHNLAHSLKSCFSYCAVFPKDHKIEKEMLIDLWIAQGYVVPLDGGQSIEDAAEEHFVILLRRCFFQDVVKDVYGDVNSVKIHDLMHDVAQEVGREEICIVNANTKNLGDKIRHVHCDVNRYAQRVSLCSHKIRSYIGGKCEKRWVDTLIDKWMCLRVLDLSRSDVKNLPNSIGKLLHLRYLNLSNNRNLKILPDAITRLHNLQTLLLEDCRSLMELPKDFCKLVKLRHLDLWGCDDLIGMPLGMDMLTSLRVLPYFVVGRKKQSVDDELKALKGLTEIKGDIRIRICSNYRIVEGMNDTGGAGYLKSMKHLTGVDITFDGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLIIIGNHGINKVMRLSGRTGLEHFTLLDSLKLSNIEDQEDEGEDNIIFWKSFPQNLRSLRIKDSDKMTSLPMGMQYLTSLQTLELSYCDELNSLPEWISSLSSLQYLRIYNCPALKSLPEAMRNLTSLQTLGG

SEQ ID NO: 37 - protein, 8 aa, Artificial Sequence
  Synthetic Construct
KDHKIEKE

SEQ ID NO: 38 - protein, 10 aa, Artificial Sequence
  Synthetic Construct
LSNNRNLKIL

SEQ ID NO: 39 - protein, 8 aa, Artificial Sequence
  Synthetic Construct
  VARIANT 4: Xaa = Lys or Glu, preferably Lys
  VARIANT 6: Xaa = Lys or Glu, preferably Glu
KDHXIXKE

SEQ ID NO: 40 - protein, 15 aa, Artificial Sequence
  Synthetic Construct
CAVFPKDHKIEKEML

SEQ ID NO: 41 - protein, 15 aa, Artificial Sequence
  Synthetic Construct
CAVFPKDHEIKKEML

SEQ ID NO: 42 - protein, 16 aa, Artificial Sequence
  Synthetic Construct
LNLSNNRNLKILPDAI

SEQ ID NO: 43 - protein, 16 aa, Artificial Sequence
  Synthetic Construct
LNLSYNEDLLMLPDAI

SEQ ID NO: 44 - protein, 16 aa, Artificial Sequence
  Synthetic Construct
LNLSYNKDLLILPDAI

SEQ ID NO: 45 - protein, 16 aa, Artificial Sequence
  Synthetic Construct
CAVFPKDHKIEKEMLI

SEQ ID NO: 46 - protein, 16 aa, Artificial Sequence
  Synthetic Construct
LNLSNNRNLKILPDAI

(19) United States
(12) Patent Application Publication
(10) Pub. No.: US 2022/0248620 A1
(43) Pub. Date: Aug. 11, 2022

(54) DOWNY MILDEW RESISTANT SPINACH AND GENES CONFERRING RESISTANCE TO DOWNY MILDEW

(71) Applicant: Enza Zaden Beheer B.V., Enkhuizen (NL)

(72) Inventors: Mathieu André PEL, Enkhuizen (NL); Faira SUIDGEEST, Enkhuizen (NL); Jan Ane DIJKSTRA, Enkhuizen (NL)

(73) Assignee: Enza Zaden Beheer B.V., Enkhuizen (NL)

(21) Appl. No.: 17/614,203

(22) PCT Filed: May 20, 2020
(86) PCT No.: PCT/EP2020/064060

§ 371 Date: Nov. 24, 2021

(30) Foreign Application Priority Data
May 24, 2019 (EP) PCTEP2019063449

(51) Int. Cl. A01H 1/00; A01H 5/10; A01H 5/12; A01H 6/02; C07K 14/415; C12N 15/82

(52) U.S. Cl. CPC A01H 1/1255; A01H 5/10; C12N 15/8216; A01H 6/028; C07K 14/415; A01H 5/12

(57) ABSTRACT

The present disclosure relates to spinach plants that are resistant to downy mildew caused by Peronospora farinosa (Pfs). The present disclosure further relates to a resistance gene that confers resistance to multiple races of Pfs in spinach plants. In addition, the 5 present disclosure relates to methods for obtaining a spinach plant that is resistant to downy mildew.

### CROSS-REFERENCE TO RELATED APPLICATIONS

This application is a U.S. National Phase application under 35 U.S.C. § 371 of International Application No. PCT/EP2020/064060, filed May 20, 2020, which claims priority to International Application No. PCT/EP2019/063449, filed May 24, 2019, each of which is incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 701802017400SEQLIST.TXT, date recorded: Nov. 8, 2021, size: 219,008 bytes).

### FIELD

The present disclosure relates to spinach plants that are resistant to downy mildew caused by Peronospora farinosa (Pfs). The present disclosure further relates to a resistance gene that confers resistance to multiple races of Pfs in spinach plants. In addition, the present disclosure relates to methods for obtaining a spinach plant that is resistant to downy mildew.

### BACKGROUND

Spinach (Spinacia oleracea) is an open field crop grown in many diverse environments. Spinach is a diploid crop that grows well in areas that have cool, wet springs, cool summers, and dry autumns. Optimal soil conditions for growing spinach include well-drained soils and a pH above 6. Nowadays, spinach breeding mainly focuses on disease resistance (e.g., resistance to downy mildew), crop yield, and improved nutritional value.

Breeding and screening activities help to select spinach varieties in the main production regions, where local market adaptation and dynamic resistance are important factors for success. Spinach breeding programs aim to provide varieties for all market segments: the fresh (babyleaf) market, the bunching market, as well as the frozen and canned products market. Several specific varieties of spinach are available within the main types: smooth, savoy, and oriental types. The spinach market is growing rapidly worldwide and much research is being performed to improve spinach genetics. Specific goals of spinach genetic improvements are improving disease resistance, reducing the need for biochemicals or pesticides, and improving both crop yield and crop quality. Further goals of the breeding programs are spinach varieties with broad resistance to downy mildew caused by Peronospora farinosa, which ideally already take future strains into account.

Downy mildew refers to several types of oomycetes that are parasites of plants. Downy mildew can originate from various species, but the main downy mildew genera are Peronospora, Plasmopara, and Bremia. Downy mildew is a problem in many food crops, and is one of the most problematic diseases in spinach. In spinach, downy mildew is caused by Peronospora farinosa sp. (Pfs), and this pathogen affects the production of this crop worldwide. Disease is spread from plant to plant by airborne spores. Spinach infected with downy mildew shows symptoms of discoloured areas and irregular yellow patches on upper leaf surfaces in combination with white, grey or purple mold located on the lower leaf surface. The lesions may eventually dry out and turn brown.

Fungicides can be used to control Peronospora farinosa, but eventually Peronospora farinosa becomes immune to these chemicals, because over time the pathogen also acquires resistance to fungicides. In addition, the market wishes to reduce the use of such chemicals in the production of food crops. Therefore, it is of the utmost importance to find other methods to control Peronospora farinosa infection. The most preferable form of control would be a resistance gene that provides broad resistance against Peronospora farinosa. Also, one or more resistance genes (e.g., with narrower resistance) can be combined to achieve broad and durable resistance against Peronospora farinosa. Therefore, identification of new resistance genes is a promising alternative to chemical control.

Seventeen official races of Peronospora farinosa have been identified to date (Pfs1 to Pfs17). Characterization of these races is based on qualitative disease reactions on a specific set of different hosts (differentials), which is an approach widely used to identify races of many plant pathogens. For spinach, the current set of differentials contains new and old commercial hybrids as well as open-pollinated cultivars and breeding lines (NIL lines). This range of differentials is required because downy mildew in spinach is particularly complex, and rapidly evolves to evade disease resistance. Under the pressure of disease resistance genes, the pathogen mutates to break down the disease resistance, meaning that new disease resistance in crops is needed to control infection. There are many different races of downy mildew, and new resistant downy mildew races, i.e., races that break current spinach resistances, emerge all the time. Breakthrough can occur as quickly as within 4 to 6 months of a new spinach resistance being developed. The main problem is that the present spinach varieties on the market, which combine different resistances, become outdated very fast as Peronospora farinosa quickly evolves new virulent races. With new races of Peronospora farinosa popping up in spinach over the last several years, it has become increasingly more difficult to stay a step ahead of the devastating disease.

At present there is no single resistance gene available that provides full spectrum resistance to all races of Peronospora farinosa. Therefore, it is an advantage to combine or stack multiple resistance genes into a spinach plant, such that a plant is obtained that includes multiple resistance genes and is resistant to all Peronospora farinosa races, or at least is resistant to as many Peronospora farinosa races as possible.

Considering the above, there is a need in the art to develop a more diverse and durable resistance in spinach and to provide spinach plants that are resistant to downy mildew caused by Peronospora farinosa. In particular, there is a need to provide spinach plants that have a broad spectrum resistance against Peronospora farinosa. Furthermore, it is an object of present disclosure to provide a method to obtain such downy mildew resistant plants. There is a need for more diversity of genes, so that more genetic variation can be achieved in commercial hybrids, making it harder for pathogens such as Peronospora farinosa to adapt. The broader the resistance of these genes, the more effectively they can be used in the development of resistant plants.

### BRIEF SUMMARY

It is an object of the present disclosure, amongst other objects, to address the above need in the art. The object of present disclosure, amongst other objects, is met by the present disclosure as outlined in the appended claims.

Specifically, the above object, amongst other objects, is met, according to a first aspect, by the present disclosure by a spinach plant that is resistant to downy mildew caused by Peronospora farinosa (Pfs), wherein the spinach plant comprises one or more resistance genes, wherein said one or more resistance genes encode for a protein having at least 85% sequence identity with SEQ ID No. 4, preferably at least 90%, more preferably at least 95%, even more preferably at least 98%, most preferably 100%, wherein the protein comprises a conserved amino acid sequence KDHKIEKE (SEQ ID NO: 37) and a conserved amino acid sequence LSNNRNLKIL (SEQ ID NO: 38). The identification of novel candidate dominant Pfs resistance genes of the present disclosure are also known as CC-NBS-LLR genes. Novel resistance genes were found, more specifically T10, T70, T71, T72, T75, T76, T83, T89, T96, T253, T18, T133, T139, T170 and T175 were obtained by sequencing of locus 1 and gene mapping of Peronospora farinosa resistance genes in Spinach. Preferably, the spinach plant of the present disclosure comprises at least two of the novel resistance genes selected from the group T10, T70, T71, T72, T75, T76, T83, T89, T96, T253, T18, T133, T139, T170, or T175.

In spinach these novel resistance genes were mapped on locus 1 on chromosome 2 in the spinach genome. The similarity of the novel Pfs resistance genes was determined using multiple alignment software and showed to be highly conserved (See Table 1). The coding sequences of the novel Pfs resistance genes showed a sequence similarity of at least ˜94%. The coding sequence of T10 is represented by SEQ ID No.1, T70 is represented by SEQ ID No.3, T71 is represented by SEQ ID No.5, T72 is represented by SEQ ID No.7, T75 is represented by SEQ ID No.9, T76 is represented by SEQ ID No.11, T83 is represented by SEQ ID No.13, T89 is represented by SEQ ID No.15, T96 is represented by SEQ ID No. 23, T253 is represented by SEQ ID No.25, T18 is represented by SEQ ID No.27, T133 is represented by SEQ ID No.29, T139 is represented by SEQ ID No.31, T170 is represented by SEQ ID No.33, and T175 is represented by SEQ ID No.35.

According to a preferred embodiment, the present disclosure relates to the spinach plant wherein said one or more resistance genes encode for a protein, wherein said protein is selected from the group consisting of SEQ ID No.2, SEQ ID No.4, SEQ ID No.6, SEQ ID No.8, SEQ ID 10, SEQ ID No.12, SEQ ID No.14, SEQ ID No.16, SEQ ID No.24, SEQ ID No.26, SEQ ID No.28, SEQ ID No.30, SEQ ID No.32, SEQ ID No.34, and SEQ ID No.36. The amino acid sequence of T10 is represented by SEQ ID No.2, T70 is represented by SEQ ID No.4, T71 is represented by SEQ ID No.6, T72 is represented by SEQ ID No.8, T75 is represented by SEQ ID No.10, T76 is represented by SEQ ID No.12, T83 is represented by SEQ ID No.14, T89 is represented by SEQ ID No.16, T96 is represented by SEQ ID No.24, T253 is represented by SEQ ID No.26, T18 is represented by SEQ ID No.28, T133 is represented by SEQ ID No.30, T139 is represented by SEQ ID No.32, T170 is represented by SEQ ID No.34, and T175 is represented by SEQ ID No.36. According to another preferred embodiment, the present disclosure relates to the spinach plant wherein the one or more genes comprise a coding sequence selected from the group consisting of SEQ ID No.1, SEQ ID No.3, SEQ ID No.5, SEQ ID No.7, SEQ ID No.9, SEQ ID No.11, SEQ ID No.13, SEQ ID No.15, SEQ ID No.23, SEQ ID No.25, SEQ ID No.27, SEQ ID No.29, SEQ ID No.31, SEQ ID No.33, and SEQ ID No.35.

According to a preferred embodiment, the present disclosure relates to the spinach plant wherein the one or more resistance genes encode for a protein, wherein the protein comprises an amino acid sequence KDHxIzKE (SEQ ID NO: 39), wherein x is amino acid K or E, preferably K, and wherein z is amino acid K or E, preferably E. The amino acid sequence KDHxIzKE (SEQ ID NO: 39) preferably corresponds to an amino acid position between 429 and 449 in the protein.

According to another preferred embodiment, the present disclosure relates to the spinach plant wherein the one or more resistance genes encode for a protein, wherein the protein comprises an amino acid sequence LSNNRNLKIL (SEQ ID NO: 38). The amino acid sequence LSNNRNLKIL (SEQ ID NO: 38) preferably corresponds to an amino acid position between 592 to 612 in the protein.

According to yet another preferred embodiment, the present disclosure relates to the spinach plant wherein said resistance genes encode for a protein, wherein said protein is comprised of an amino acid sequence KDHKIEKE (SEQ ID NO: 37) and/or an amino acid sequence LSNNRNLKIL (SEQ ID NO: 38). The proteins of the novel Pfs resistance genes of present disclosure share at least one conserved amino acid sequence at a specified position within the protein, KDHKIEKE (SEQ ID NO: 37) and/or LSNNRNLKIL (SEQ ID NO: 38).

The present disclosure generally relates to plants having one or more resistance genes, e.g. plants having an R gene encoding a NBS-LRR protein (also known as NLRs) with a CC motif in the amino-terminal domain. NLRs have a distinct domain architecture that consists of a nucleotide-binding (NB-ARC) domain and a series of C-terminal leucine-rich repeats (LRRs), and most have an N-terminal extension consisting of a Toll/interleukin-1 receptor (TIR) domain, a coiled-coil domain (CC), or a divergent coiled-coil domain (CCR). NLRs can bind and recognize effectors or recognize the modification of another plant component through its effector function. The KDHKIEKE (SEQ ID NO: 37) motif in the proteins of the resistant plants of present invention is located in the NB-ARC domain of these proteins, a nucleotide-binding adaptor shared by other R proteins, and proteins such as APAF-1 and CED-4, i.e. cytoplasmic proteins involved in the apoptosis regulatory network. It is hypothesized that the NB-ARC domain is able to bind and hydrolyse ATP. ADP binding has been experimentally verified. It is proposed that binding and hydrolysis of ATP by this domain induces conformational changes in the overall protein, leading to formation of the apoptosome. Shared domains and common evolutionary origin between NLRs (high sequence homology) suggest that multimerization through the NB-ARC domain following exchange of ADP for ATP is a key step in NLR activation and serve as such as molecular switches in immune signaling of the plant. The ADP-bound state is thought of as the “off state”, in which the LRR associates with the NB-ARC domain, thereby stabilizing the NLR in the inactive state. The activation of NLRs is generally associated with the ATP-bound state and is referred to as the “on state”. Preferably, the KDHKIEKE (SEQ ID NO: 37) motif is found between amino acid 433 and 442 in the proteins encoded by the resistance genes included in the present invention.

The LSNNRNLKIL (SEQ ID NO: 38) motif is located in one of the LRR (leucine rich repeat) domains of the protein. The primary function of these motifs appears to be to provide a versatile structural framework for the formation of protein-protein interactions. The diversification of NLRs through recombination and gene conversion generate various LRR regions that are capable of recognizing highly variable effectors and can provide resistance against pathogens. It is believed that those domains determine effector recognition and are therefore engaged in direct effector interaction and disease susceptibility/resistance. Preferably, the LSNNRNLKIL (SEQ ID NO: 38) motif is found between amino acid 596 and 607 in the proteins encoded by the resistance genes included in present invention.

According to a preferred embodiment, the present disclosure relates to the spinach plant wherein said spinach plant comprises the one or more resistance gene(s) selected from the group consisting of SEQ ID No.3, SEQ ID No.5, SEQ ID No.7, and SEQ ID No.15. T70 is represented by SEQ ID No.3, T71 is represented by SEQ ID No.5, T72 is represented by SEQ ID No.7, and T89 is represented by SEQ ID No.15.

According to yet another preferred embodiment, the present invention relates to a spinach plant that is resistant to downy mildew caused by Peronospora farinosa (Pfs), wherein the spinach plant comprises one or more resistance genes comprising a coding sequence selected from the group consisting of SEQ ID No.1, SEQ ID No.3, SEQ ID No.5, SEQ ID No.7, SEQ ID No.9, SEQ ID No.11, SEQ ID No.13, SEQ ID No.15, SEQ ID No.23, SEQ ID No.25, SEQ ID No.27, SEQ ID No.29, SEQ ID No.31, SEQ ID No.33, and SEQ ID No.35. These one or more resistance genes encode for a protein selected from the group consisting of SEQ ID No.2, SEQ ID No.4, SEQ ID No.6, SEQ ID No.8, SEQ ID 10, SEQ ID No.12, SEQ ID No.14, SEQ ID No.16, SEQ ID No.24, SEQ ID No.26, SEQ ID No.28, SEQ ID No.30, SEQ ID No.32, SEQ ID No.34, and SEQ ID No.36.

According to another preferred embodiment, the present disclosure relates to the spinach plant wherein said plant is at least resistant to Peronospora farinosa races Pfs1 to Pfs4, and Pfs7 to Pfs 17. It is expected that the spinach plant will also be resistant to Pfs 6.

According to another preferred embodiment, the present disclosure relates to the spinach plant wherein said one or more resistance genes is derived from deposit number NCIMB 43360. Seeds of Spinacia oleracea plant according to the present inventions were deposited on 21 Feb. 2019 at NCIMB Ltd, Ferguson Building, Craibstone Estate Bucksburn, AB21 9YA Aberdeen, United Kingdom.

The present disclosure, according to a second aspect, relates to seed produced by or obtained from a spinach plant according to the present disclosure, the seed comprising one or more resistance genes, wherein said one or more resistance genes encode for a protein having at least 85% sequence identity with SEQ ID No. 4, wherein the protein comprises a conserved amino acid sequence KDHKIEKE (SEQ ID NO: 37) and a conserved amino acid sequence LSNNRNLKIL (SEQ ID NO: 38).

The present disclosure, according to a third aspect, relates to a resistance gene that confers resistance to downy mildew in spinach plants, wherein the gene encodes for a protein that has at least 85% sequence identity with SEQ ID No. 4, preferably at least 90%, more preferably at least 95%, even more preferably at least 98%, most preferably 100%. The novel resistance genes encode for proteins that confer broad Pfs resistance in spinach. The coding sequence of the resistance gene has at least 90% sequence identity with SEQ ID No. 3, preferably at least 94%, more preferably at least 98%, even more preferably at least 99%, most preferably 100%.

According to a preferred embodiment, the present disclosure relates to the resistance gene, wherein the gene comprises a coding sequence selected from the group consisting of SEQ ID No.1., SEQ ID No.3., SEQ ID No.5., SEQ ID No.7., SEQ ID No.9., SEQ ID No.11., SEQ ID No.13., SEQ ID No.15, SEQ ID No.23, SEQ ID No.25, SEQ ID No.27, SEQ ID No.29, SEQ ID No.31, SEQ ID No.33, and SEQ ID No.35.

According to yet another preferred embodiment, the present disclosure relates to the resistance gene, wherein the resistance gene encodes for a protein, wherein the protein comprises an amino acid sequence KDHxIzKE (SEQ ID NO: 39), wherein x is amino acid K or E, preferably K, and wherein z is amino acid K or E, preferably E.

According to a preferred embodiment, the present disclosure relates to the resistance gene, wherein the resistance gene encodes for a protein, wherein the protein comprises a conserved amino acid sequence conserved amino acid sequence LSNNRNLKIL (SEQ ID NO: 38).

According to yet another preferred embodiment, the present disclosure relates to the resistance gene, wherein the resistance gene encodes for a protein, wherein said protein is comprised of an amino acid sequence KDHKIEKE (SEQ ID NO: 37) and/or an amino acid sequence LSNNRNLKIL (SEQ ID NO: 38).

According to another preferred embodiment, the present disclosure relates to the resistance gene, wherein the coding sequence of said resistance gene is selected from the group consisting of SEQ ID No.3, SEQ ID No.5, SEQ ID No.7, and SEQ ID No.15, and provides at least resistance to Peronospora farinosa races Pfs1 to Pfs4, and Pfs7 to Pfs 17 in spinach. Preferably the resistance gene is SEQ ID No.7, more preferably SEQ ID No.5, even more preferably SEQ ID No.3, and most preferably SEQ ID No.15.

According to a further aspect, the present disclosure relates to a method for providing a spinach plant that is resistant to downy mildew, wherein the method comprises the steps of introducing or modifying one or more resistance genes into the genome of the spinach plant, wherein the one or more resistance genes are selected from the group consisting of SEQ ID No.1, SEQ ID No.3, SEQ ID No.5, SEQ ID No.7, SEQ ID No.9, SEQ ID No.11, SEQ ID No.13, SEQ ID No.15, SEQ ID No.23, SEQ ID No.25, SEQ ID No.27, SEQ ID No.29, SEQ ID No.31, SEQ ID No.33, and SEQ ID No.35.

According to another preferred embodiment, the present disclosure relates to the method, wherein the introduction or modification of the one or more Pfs resistance genes is achieved by genome editing techniques, CRISPR Cas, or mutagenesis techniques.

The present disclosure, according to a further aspect, relates to a method for providing a spinach plant that is resistant to downy mildew, wherein the method comprises the steps of:

a) providing a spinach plant comprising one or more resistance gene(s), of the present disclosure,
b) crossing the spinach plant of step a) with a susceptible spinach plant,
c) optionally, selfing the plant obtained in step b) for at least one time, and
d) selecting the plants that are resistant to downy mildew.

According to a preferred embodiment, the present disclosure relates to the method, wherein the coding sequence of said one or more resistance genes is selected from the group consisting of SEQ ID No.3, SEQ ID No.5, SEQ ID No.7, and SEQ ID No.15.

According to another preferred embodiment, the present disclosure relates to the method, wherein the spinach plant is resistant to downy mildew caused by Peronospora farinosa races Pfs1 to Pfs4, and Pfs7 to Pfs 17.

According to a preferred embodiment, the present disclosure relates to the method, wherein the one or more resistance gene(s) is obtained from deposit number NCIMB 43360.

### BRIEF DESCRIPTION OF THE DRAWINGS

The present invention will be further detailed in the following examples and figures wherein:

FIG. 1 shows qPCR quantification shows quantification of Pfs actin in spinach plants infected with Pfs race 14 (Pfs 14), after VIGS gene silencing. The spinach plants used for the quantification were spinach plants that were not transformed with a VIGS construct (“Non-treated”), resistant spinach plants containing the T70 gene that were transiently transformed with a RFP VIGS silencing construct (“VIGS RFP”; negative control), and resistant spinach plants containing the T70 gene that were transiently transformed with a T70 VIGS silencing construct (“VIGS T70”). Three technical replicates were performed. In case T70 gene expression levels were VIGS silenced in spinach infected with Pfs 14, expression levels of Pfs actin increased dramatically. Leaves of the plant that were susceptible to Pfs, showed high transcriptional levels of the Pfs actin housekeeping gene, indicating the susceptibility corresponds with low T70 gene expression due to VIGS silencing.

FIG. 2 shows alignments of the two conserved amino acid sequence motifs in the proteins encoded by the resistance genes T10, T70, T71, T72, T75, T76, T83, and T89. The first alignment (shown at the top of FIG. 2) shows T10 (SEQ ID NO: 40), T70 (SEQ ID NO: 40), T71 (SEQ ID NO: 40), T72 (SEQ ID NO: 40), T75 (SEQ ID NO: 40), T76 (SEQ ID NO: 41), T83 (SEQ ID NO: 40), and T89 (SEQ ID NO: 40). All of these proteins contain a first conserved amino acid sequence motif KDHxIzKE (SEQ ID NO: 39), which is located at a position between 429 and 449 (x=amino acid K or E, preferably K; z=K or E, preferably E). The second alignment (shown at the bottom of FIG. 2) shows T10 (SEQ ID NO: 42), T70 (SEQ ID NO: 42), T71 (SEQ ID NO: 42), T72 (SEQ ID NO: 42), T75 (SEQ ID NO: 43), T76 (SEQ ID NO: 44), T83 (SEQ ID NO: 42), and T89 (SEQ ID NO: 42). Most of these proteins also contain a second conserved amino acid sequence motif LSNNRNLKIL (SEQ ID NO: 38), approximately at position 592 to 612.

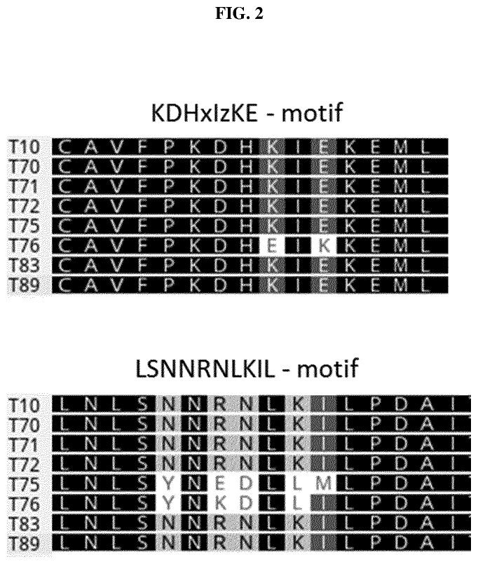

FIG. 3 shows alignments of the two conserved amino acid sequence motifs in the proteins encoded by the resistance genes T10, T70, T71, T72, T83, T89, T96, T253, T18, T133, T139, T170 and T175. The first alignment (shown at the top of FIG. 3) shows T10 (SEQ ID NO: 45), T70 (SEQ ID NO: 45), T71 (SEQ ID NO: 45), T72 (SEQ ID NO: 45), T83 (SEQ ID NO: 45), T89 (SEQ ID NO: 45), T96 (SEQ ID NO: 45), T253 (SEQ ID NO: 45), T18 (SEQ ID NO: 45), T133 (SEQ ID NO: 45), T139 (SEQ ID NO: 45), T170 (SEQ ID NO: 45), and T175 (SEQ ID NO: 45). All of these proteins contain a first conserved amino acid sequence motif KDHKIEKE (SEQ ID NO: 37). The second alignment (shown at the bottom of FIG. 3) shows T10 (SEQ ID NO: 46), T70 (SEQ ID NO: 46), T71 (SEQ ID NO: 46), T72 (SEQ ID NO: 46), T83 (SEQ ID NO: 46), T89 (SEQ ID NO: 46), T96 (SEQ ID NO: 46), T253 (SEQ ID NO: 46), T18 (SEQ ID NO: 46), T133 (SEQ ID NO: 46), T139 (SEQ ID NO: 46), T170 (SEQ ID NO: 46), and T175 (SEQ ID NO: 46). All of these proteins contain a second conserved amino acid sequence motif LSNNRNLKIL (SEQ ID NO: 38).

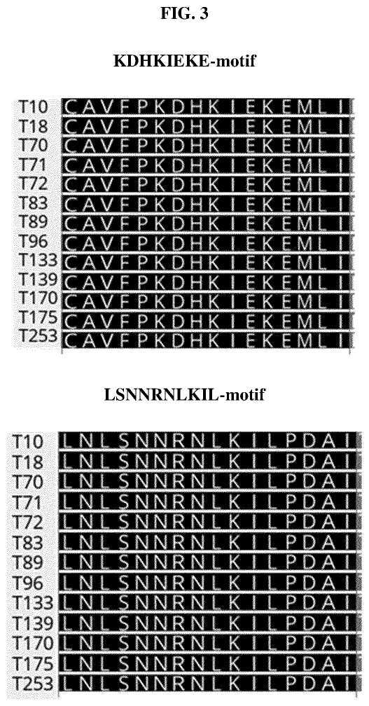

### DETAILED DESCRIPTION

### Disease Resistance Genes and Proteins

Nucleotide-binding site leucine-rich repeat proteins, also known as NBS-LRR proteins, are encoded by disease resistance genes in plants known as R genes. NBS-LRR proteins are characterized by nucleotide-binding site (NBS) and leucine-rich repeat (LRR) domains as well as variable amino- and carboxy-terminal domains. These proteins are involved in the detection of diverse pathogens, including bacteria, viruses, fungi, nematodes, insects, and oomycetes. There are two major subfamilies of plant NBS-LRR proteins, which are defined by the Toll/interleukin-1 receptor (TIR) or the coiled-coil (CC) motifs in their amino-terminal domains, and proteins of both subfamilies are involved in pathogen recognition.

Most known resistances in spinach were identified from a locus, which is called Locus 1 and is located on chromosome 2 (LG2) and is highly variable. Although many genes have been identified in many different wild spinach accessions, for most genes, it is still unknown whether they are functional (e.g., provide downy mildew resistance) or not.

The present disclosure generally relates to plants having one or more resistance genes, e.g. plants having an R gene encoding a NBS-LRR protein with a TIR motif in the amino-terminal domain. In some embodiments, having one or more resistance genes provides broad spectrum resistance to downy mildew (e.g., Peronospora farinosa). In some embodiments, having one or more resistance genes provides resistance to at least fifteen races of Peronospora farinosa.

In some aspects, plants of the present disclosure are Spinacea oleracea, also known as spinach. Spinach contains many resistance genes, known as R genes. In particular, spinach contains an R gene that originates from Locus 1. In some aspects, plants of the present disclosure have resistance genes that are present in seeds deposited under accession number NCIMB 43360.

Certain aspects of the present disclosure relate to a resistance gene with the nucleotide coding sequence SEQ ID NO: 1. Provided herein are also homologs and orthologs of SEQ ID NO: 1. In some embodiments, a homolog or ortholog of SEQ ID NO: 1 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1.

Certain aspects of the present disclosure relate to a resistance gene with the nucleotide coding sequence SEQ ID NO: 3. Provided herein are also homologs and orthologs of SEQ ID NO: 3. In some embodiments, a homolog or ortholog of SEQ ID NO: 3 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 3.

Certain aspects of the present disclosure relate to a resistance gene with the nucleotide coding sequence SEQ ID NO: 5. Provided herein are also homologs and orthologs of SEQ ID NO: 5. In some embodiments, a homolog or ortholog of SEQ ID NO: 5 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 5.

Certain aspects of the present disclosure relate to a resistance gene with the nucleotide coding sequence SEQ ID NO: 7. Provided herein are also homologs and orthologs of SEQ ID NO: 7. In some embodiments, a homolog or ortholog of SEQ ID NO: 7 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 7.

Certain aspects of the present disclosure relate to a resistance gene with the nucleotide coding sequence SEQ ID NO: 9. Provided herein are also homologs and orthologs of SEQ ID NO: 9. In some embodiments, a homolog or ortholog of SEQ ID NO: 9 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 9.

Certain aspects of the present disclosure relate to a resistance gene with the nucleotide coding sequence SEQ ID NO: 11. Provided herein are also homologs and orthologs of SEQ ID NO: 11. In some embodiments, a homolog or ortholog of SEQ ID NO: 11 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 11.

Certain aspects of the present disclosure relate to a resistance gene with the nucleotide coding sequence SEQ ID NO: 13. Provided herein are also homologs and orthologs of SEQ ID NO: 13. In some embodiments, a homolog or ortholog of SEQ ID NO: 13 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 13.

Certain aspects of the present disclosure relate to a resistance gene with the nucleotide coding sequence SEQ ID NO: 15. Provided herein are also homologs and orthologs of SEQ ID NO: 15. In some embodiments, a homolog or ortholog of SEQ ID NO: 15 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 15.

In some aspects, plants of the present disclosure have a resistance gene with the nucleotide coding sequence SEQ ID NO: 1. In some embodiments, these plants may also have one or more resistance genes with nucleotide coding sequences selected from the group of SEQ ID NO: 3, SEQ ID NO: 5, SEQ ID NO: 7, SEQ ID NO: 9, SEQ ID NO: 11, SEQ ID NO: 13, or SEQ ID NO: 15.

Certain aspects of the present disclosure relate to a resistance protein with the amino acid sequence SEQ ID NO: 2. Provided herein are also homologs and orthologs of SEQ ID NO: 2. In some embodiments, a homolog or ortholog of SEQ ID NO: 2 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 2.

Certain aspects of the present disclosure relate to a resistance protein with the amino acid sequence SEQ ID NO: 4. Provided herein are also homologs and orthologs of SEQ ID NO: 4. In some embodiments, a homolog or ortholog of SEQ ID NO: 4 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 4.

Certain aspects of the present disclosure relate to a resistance protein with the amino acid sequence SEQ ID NO: 6. Provided herein are also homologs and orthologs of SEQ ID NO: 6. In some embodiments, a homolog or ortholog of SEQ ID NO: 6 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 6.

Certain aspects of the present disclosure relate to a resistance protein with the amino acid sequence SEQ ID NO: 8. Provided herein are also homologs and orthologs of SEQ ID NO: 8. In some embodiments, a homolog or ortholog of SEQ ID NO: 8 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 8.

Certain aspects of the present disclosure relate to a resistance protein with the amino acid sequence SEQ ID NO: 10. Provided herein are also homologs and orthologs of SEQ ID NO: 10. In some embodiments, a homolog or ortholog of SEQ ID NO: 10 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 10.

Certain aspects of the present disclosure relate to a resistance protein with the amino acid sequence SEQ ID NO: 12. Provided herein are also homologs and orthologs of SEQ ID NO: 12. In some embodiments, a homolog or ortholog of SEQ ID NO: 12 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12.

Certain aspects of the present disclosure relate to a resistance protein with the amino acid sequence SEQ ID NO: 14. Provided herein are also homologs and orthologs of SEQ ID NO: 14. In some embodiments, a homolog or ortholog of SEQ ID NO: 14 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 14.

Certain aspects of the present disclosure relate to a resistance protein with the amino acid sequence SEQ ID NO: 16. Provided herein are also homologs and orthologs of SEQ ID NO: 16. In some embodiments, a homolog or ortholog of SEQ ID NO: 16 has a coding sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 16.

In some aspects, plants of the present disclosure have a resistance protein with the amino acid sequence SEQ ID NO: 2. In some embodiments, these plants may also have one or more resistance proteins with amino acid sequences selected from the group of SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID No.24, SEQ ID No.26, SEQ ID No.28, SEQ ID No.30, SEQ ID No.32, SEQ ID No.34 and SEQ ID No.36.

In some aspects, plants of the present disclosure have a resistance protein containing one or more or two amino acid consensus motifs. In some embodiments, the resistance protein has a first amino acid consensus motif of KDHxIzKE (SEQ ID NO: 39), wherein x is amino acid K or E, preferably K, and wherein z is amino acid K or E, preferably E. In some embodiments, the first amino acid consensus motif is KDHKIEKE (SEQ ID NO: 37). In some embodiments, the resistance protein has a second amino acid consensus motif of LSNNRNLKIL (SEQ ID NO: 38). In some embodiments, the resistance protein has both a first amino acid consensus motif and a second amino acid consensus motif.

The first amino acid consensus motif KDHxIzKE (SEQ ID NO: 39) (e.g., KDHKIEKE (SEQ ID NO: 37)) is located in the NB-ARC domain of the protein, which is a nucleotide-binding adaptor shared by other R proteins, and proteins such as APAF-1 and CED-4 (i.e., cytoplasmic proteins involved in the apoptosis regulatory network). It is hypothesized that the NB-ARC domain is able to bind and hydrolyse ATP. ADP binding has been experimentally verified. It is proposed that binding and hydrolysis of ATP by the NB-ARC domain induces conformational changes in the overall protein, leading to formation of the apoptosome.

The second amino acid consensus motif LSNNRNLKIL (SEQ ID NO: 38) is located in one of the LRR (leucine rich repeat) domains of the protein. The primary function of these motifs appears to be to provide a versatile structural framework for the formation of protein-protein interactions. It is thought that those domains determine effector recognition and therefore disease susceptibility/resistance.

Resistance to Peronospora farinosa

The present disclosure generally relates to plants having a resistance to downy mildew (e.g., Peronospora farinosa) resistance. In some embodiments, plants of the present disclosure have broad spectrum resistance to Peronospora farinosa. In some embodiments, plants of the present disclosure are resistant to fifteen or more, sixteen or more, or seventeen or more races of Peronospora farinosa. In some embodiments, plants of the present disclosure are resistant to fifteen or more, sixteen or more, or seventeen races of Peronospora farinosa selected from the group of Pfs1, Pfs2, Pfs3, Pfs4, Pfs5, Pfs6, Pfs7, Pfs8, Pfs9, Pfs10, Pfs11, Pfs12, Pfs13, Pfs14, Pfs15, Pfs16, or Pfs17. In some embodiments, plants of the present disclosure have resistance to Pfs1, Pfs2, Pfs3, Pfs4, Pfs7, Pfs8, Pfs9, Pfs10, Pfs11, Pfs12, Pfs13, Pfs14, Pfs15, Pfs16, and Pfs17. In some embodiments, plants of the present disclosure additionally have resistance to other races of Peronospora farinosa.

In some embodiments, the presence of one or more coding sequences of one or more resistance genes results in Peronospora farinosa resistance. In some embodiments, the one or more coding sequences are selected from the group of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID No.23, SEQ ID No.25, SEQ ID No.27, SEQ ID No.29, SEQ ID No.31, SEQ ID No.33 and SEQ ID No.35. In some embodiments, the one or more coding sequences are selected from the group of SEQ ID NO: 3, SEQ ID NO: 5, SEQ ID NO: 7, or SEQ ID NO: 15.

In some embodiments, the presence of one or more resistance proteins results in Peronospora farinosa resistance. In some embodiments, the one or more resistance proteins is selected from the group of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID No.24, SEQ ID No.26, SEQ ID No.28, SEQ ID No.30, SEQ ID No.32, SEQ ID No.34 and SEQ ID No.36. In some embodiments, the resistance protein is SEQ ID NO: 4.

### Plants of the Present Disclosure

In some aspects, plants of the present disclosure are plants of the family Amaranthaceae. In some embodiments, plants of the present disclosure are plants of the species Spinacia oleracea (spinach).

According to the present description plant parts include, but are not limited to, leaves, stems, meristems, cotyledons, hypocotyl, roots, root tips, root meristems, ovules, pollen, anthers, pistils, flowers, embryos, seeds, fruits, parts of fruits, cells, and the like. Plant tissues may be tissues or any plant part. Plant cells may be cells of any plant part.

Plants of the present disclosure include plants with resistance genes having coding sequences selected from the group of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID No.23, SEQ ID No.25, SEQ ID No.27, SEQ ID No.29, SEQ ID No.31, SEQ ID No.33, and SEQ ID No.35. In some embodiments, plants of the present disclosure include plants with resistance genes having coding sequences selected from the group of SEQ ID NO: 3, SEQ ID NO: 5, SEQ ID NO: 7, or SEQ ID NO: 15.

Plants of the present disclosure include plants with resistance proteins having amino acid sequences selected from the group of SEQ ID NO: 2, SEQ ID NO:4, SEQ ID NO: 6, SEQ ID NO: 8, SEQ ID NO: 10, SEQ ID NO:12, SEQ ID NO:14, SEQ ID NO: 16, SEQ ID No.24 SEQ ID No.26, SEQ ID No.28, SEQ ID No.30, SEQ ID No.32, SEQ ID No.34, and SEQ ID No.36. In some embodiments, plants of the present disclosures include plants with a resistance protein having amino acid sequence SEQ ID NO: 4.

Plants of the present disclosure include plants with resistance proteins containing a first conserved amino acid motif KDHxIzKE (SEQ ID NO: 39), where x is amino acid K or E, preferably K, and z is amino acid K or E, preferably E (e.g., KDHKIEKE (SEQ ID NO: 37)); a second conserved amino acid motif LSNNRNLKIL (SEQ ID NO: 38); or both a first conserved amino acid motif KDHxIzKE (SEQ ID NO: 39) and a second conserved amino acid motif LSNNRNLKIL (SEQ ID NO: 38).

Plants of the present disclosure include spinach plants grown from seeds deposited under accession number NCIMB 43360. In another embodiment, the present invention is directed to a spinach plant and parts isolated therefrom having all the physiological and morphological characteristics of a spinach plant produced by growing spinach seed having NCIMB Accession Number 43360. In still another embodiment, the present invention is directed to an F₁ hybrid spinach seed, plants grown from the seed, and leaves isolated therefrom having a spinach plant as a parent, where the spinach plant is grown from spinach seed having NCIMB Accession Number 43360. In some embodiments, one or more of the resistance genes having coding sequences selected from the group of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID No.23, SEQ ID No.25, SEQ ID No.27, SEQ ID No.29, SEQ ID No.31, SEQ ID No.33, and SEQ ID No.35 is present in plants grown from seeds deposited under accession number NCIMB 43360. In some embodiments, one or more of the resistance proteins having amino acid sequences selected from the group of SEQ ID NO: 2, SEQ ID NO:4, SEQ ID NO: 6, SEQ ID NO: 8, SEQ ID NO: 10, SEQ ID NO:12, SEQ ID NO:14, SEQ ID NO: 16, SEQ ID No.23, SEQ ID No.25, SEQ ID No.27, SEQ ID No.29, SEQ ID No.31, SEQ ID No.33, and SEQ ID No.35 is present in plants grown from seeds deposited under accession number NCIMB 43360.

In order to determine whether a plant is a plant of the present disclosure, and therefore whether said plant has the same genes as plants of the present disclosure, the phenotype of the plant can be compared with the phenotype of a known plant of the present disclosure (e.g., a plant grown from seeds deposited under accession number NCIMB 43360). In some embodiments, plants of the present disclosure have broad spectrum downy mildew (Peronospora farinosa) resistance. In some embodiments, plants of the present disclosure have resistance to fifteen or more Pfs races selected from the group of Pfs1, Pfs2, Pfs3, Pfs4, Pfs5, Pfs6, Pfs7, Pfs8, Pfs9, Pfs10, Pfs11, Pfs12, Pfs13, Pfs14, Pfs15, Pfs16, or Pfs17. In some embodiments, the phenotype can be assessed by, for example, the downy mildew leaf disc assay, as described in Example 4. In some embodiments, the phenotype can be assessed by disease resistance assays known to one of skill in the art.

In addition to phenotypic observations, the genotype of a plant can also be examined There are many laboratory-based techniques known in the art that are available for the analysis, comparison and characterization of plant genotype. Such techniques include, without limitation, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), and Single Nucleotide Polymorphisms (SNPs). By using these techniques, it is possible to assess the presence of the alleles, genes, and/or loci involved in the downy mildew resistance phenotype of the plants of the present disclosure.

In addition, the gene expression of a plant or a pathogen can be examined. There are many laboratory-based techniques known in the art that are available for the analysis, comparison, and characterization of plant or pathogen gene expression. Such techniques include, without limitation, Quantitative Polymerase Chain Reaction (qPCR; also referred to as Real-Time PCR), Reverse Transcription Polymerase Chain Reaction (RT-PCR), and RNA sequencing (RNA-Seq). For example, the expression of pathogen genes can be assessed using qPCR, and used to determine whether a plant has the downy mildew resistance phenotype of the plants of the present disclosure, as described in Example 3.

### Methods of Obtaining Plants of the Present Disclosure

In some embodiments, the present disclosure is directed to a method of selecting spinach plants, by a) growing spinach plants containing one or more resistance genes or one or more resistance proteins of the present disclosure and b) selecting a plant from step a). In some embodiments, the present disclosure is directed to a method of breeding spinach plants by crossing a spinach plant with a plant containing one or more resistance genes or one or more resistance proteins of the present disclosure. In some embodiments, the present disclosure is directed to a method of breeding a spinach plant that is resistant to downy mildew, by (a) providing a spinach plant containing one or more resistance genes or one or more resistance proteins of the present disclosure, (b) crossing the spinach plant of step (a) with a susceptible spinach plant, (c) optionally, selfing the plant obtained in step (b) at least once, and (d) selecting the plants that are resistant to downy mildew. In some embodiments, the present disclosure is directed to methods of introducing a desired trait into a spinach plant containing one or more resistance genes or one or more resistance proteins of the present disclosure, by: (a) crossing a spinach plant containing one or more resistance genes or one or more resistance proteins of the present disclosure with a plant of another spinach variety that contains a desired trait to produce progeny plants, where the desired trait is selected from herbicide resistance; insect or pest resistance; and resistance to bacterial disease, fungal disease, oomycete disease, or viral disease; (b) selecting one or more progeny plants that have the desired trait; (c) backcrossing the selected progeny plants with a spinach plant containing one or more resistance genes or one or more resistance proteins of the present disclosure to produce backcross progeny plants; (d) selecting for backcross progeny plants that have the desired trait and all of the physiological and morphological characteristics of a spinach plant containing one or more resistance genes or one or more resistance proteins of the present disclosure; and (e) repeating steps (c) and (d) two or more times in succession to produce selected third or higher backcross progeny plants that comprise the desired trait. In some embodiments, the present disclosure is directed to a method of obtaining spinach plants by growing spinach seed containing one or more resistance genes or one or more resistance proteins of the present disclosure. In some embodiments that may be combined with any of the preceding embodiments, the spinach progeny plants have broad spectrum resistance to downy mildew (Peronospora farinosa).

In some embodiments, the present disclosure is directed to a method of selecting spinach plants, by a) growing spinach plants from spinach seed having NCIMB Accession Number 43360 and b) selecting a plant from step a). In some embodiments, the present disclosure is directed to a method of breeding spinach plants by crossing a spinach plant with a plant grown from spinach seed having NCIMB Accession Number 43360. In some embodiments, the present disclosure is directed to a method of breeding a spinach plant that is resistant to downy mildew, by (a) providing a spinach plant, where a sample of spinach seed was deposited under NCIMB Accession Number 43360, (b) crossing the spinach plant of step (a) with a susceptible spinach plant, (c) optionally, selfing the plant obtained in step (b) at least once, and (d) selecting the plants that are resistant to downy mildew. In some embodiments, the present disclosure is directed to methods of introducing a desired trait into a spinach plant grown from spinach seed having NCIMB Accession Number 43360, by: (a) crossing a spinach plant, where a sample of spinach seed was deposited under NCIMB Accession Number 43360, with a plant of another spinach variety that contains a desired trait to produce progeny plants, where the desired trait is selected from herbicide resistance; insect or pest resistance; and resistance to bacterial disease, fungal disease, oomycete disease, or viral disease; (b) selecting one or more progeny plants that have the desired trait; (c) backcrossing the selected progeny plants with a spinach plant grown from spinach seed having NCIMB Accession Number 43360 to produce backcross progeny plants; (d) selecting for backcross progeny plants that have the desired trait and all of the physiological and morphological characteristics of a spinach plant grown from spinach seed having NCIMB Accession Number 43360; and (e) repeating steps (c) and (d) two or more times in succession to produce selected third or higher backcross progeny plants that comprise the desired trait. In some embodiments, the present disclosure is directed to a method of obtaining spinach plants by growing spinach seed having NCIMB Accession Number 43360. In some embodiments that may be combined with any of the preceding embodiments, the spinach progeny plants have broad spectrum resistance to downy mildew (Peronospora farinosa).

A resistance gene or protein of the present disclosure can be brought into the plant by means of breeding. The breeding technique called backcrossing allows essentially all of the desired morphological and physiological characteristics of a cultivar to be recovered in addition to the single gene transferred into the line (e.g., the resistance gene encoding a protein with amino acid sequence SEQ ID NO: 4). The parental spinach plant which contributes the gene for the desired characteristic (e.g., the resistance gene encoding a protein with amino acid sequence SEQ ID NO: 4) is termed the nonrecurrent or donor parent. This terminology refers to the fact that the nonrecurrent parent is used one time in the backcross protocol and therefore does not recur. The parental spinach plant to which the gene or genes from the nonrecurrent parent are transferred is known as the recurrent parent as it is used for several rounds in the backcrossing protocol. In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a spinach plant is obtained wherein essentially all of the desired morphological and physiological characteristics of the recurrent parent are recovered in the converted plant, in addition to the single transferred gene from the nonrecurrent parent. The present disclosure further relates to methods for developing spinach plants in a spinach plant breeding program using plant breeding techniques including recurrent selection, backcrossing, pedigree breeding, restriction fragment length polymorphism enhanced selection, and genetic marker enhanced selection.

A resistance gene or protein of the present disclosure can also be brought into the plant by means of transgenic techniques. Plant transformation involves the construction of an expression vector that will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed melon plants. Promoters may be inducible, constitutive, tissue-specific or tissue-preferred. Methods for plant transformation include biological methods and physical methods (See, for example, Miki, et al., “Procedures for Introducing Foreign DNA into Plants” in Methods in Plant Molecular Biology and Biotechnology, Glick and Thompson Eds., CRC Press, Inc., Boca Raton, pp. 67-88 (1993)). In addition, expression vectors and in vitro culture methods for plant cell or tissue transformation and regeneration of plants are available (See, for example, Gruber, et al., “Vectors for Plant Transformation” in Methods in Plant Molecular Biology and Biotechnology, Glick and Thompson Eds., CRC Press, Inc., Boca Raton, pp. 89-119 (1993)). The produced transgenic line could then be crossed, with another (non-transformed or transformed) line, in order to produce a new transgenic line. Alternatively, a genetic trait which has been engineered into a particular spinach cultivar using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene.

In some embodiments, the endogenous resistance genes can be modified or mutated using mutagenesis, gene editing techniques, or other methods known in the art to obtain the plants of the current disclosure. In some embodiments, the gene editing technique is selected from the group of transcription activator-like effector nuclease (TALEN) gene editing techniques, clustered Regularly Interspaced Short Palindromic Repeat (CRISPR/Cas9) gene editing techniques, or zinc-finger nuclease (ZFN) gene editing techniques. In some embodiments, the mutation is introduced using one or more vectors including gene editing components selected from the group of a CRISPR/Cas9 system, a TALEN, a zinc finger, and a meganuclease designed to target a nucleic acid sequence encoding a resistance gene.

Plants of the present disclosure can be identified by multiple methods, as described above. The gene expression levels can, for example, be tested by analysis of transcript levels (e.g., by RT-PCR) produced from a coding sequence of the present disclosure, such as SEQ ID NO: 3. Another option is the quantification of resistance protein levels (e.g., of the resistance protein with amino acid sequence SEQ ID NO: 4), for example by using antibodies. The skilled person can also use the usual pathogen tests to see if the downy mildew resistance is a broad spectrum downy mildew resistance. These methods are known to the person skilled in the art and can be used to identify plants of the present disclosure. Plants with the desired resistance genes or proteins are then propagated, back-crossed, or crossed to other breeding lines to transfer only the desired new gene(s) into the background of the crop wanted.

### EXAMPLES

The following examples are provided to further illustrate aspects of the present disclosure. These examples are non-limiting and should not be construed as limiting any aspect of the present disclosure.

### Example 1: Genetic Mapping to Identify Novel Candidate Dominant Resistance Genes

The novel candidate dominant resistance genes were obtained by gene mapping Peronospora farinosa resistance genes in spinach (S. oleracea). The resistance genes were mapped using a Bulked segregant analysis (BSA) approach. The RNA of multiple resistance families (originating from the F3 generation) were pooled and compared to a pool of RNA of susceptible families. All F3 families were derived from the same F2 plant. Markers were developed in regions where an increase in the number of SNPs was observed. The markers were validated using the F2 population. Once a region of interest (ROI) could be identified and flanked by markers, a fine mapping approach was started.

### Example 2: Virus Induced Gene Silencing (VIGS) of the T70 Gene in Spinach (S. oleracea)

To demonstrate that the T70 gene was related to Peronospora farinosa resistance, the putative resistance gene (T70) was silenced using tobacco rattle virus (TRV)-based virus-induced gene silencing (VIGS). This was done to see whether VIGS silencing of resistant spinach lines containing the T70 resistance gene would induce susceptibility to P. farinosa infection.

Construction of VIGS Constructs and Transformation of VIGS Constructs into Spinach (S. oleracea)

The use of TRV-derived VIGS vectors for studying gene function is well known, and VIGS vectors have been used to study gene function in multiple plant species, including Arabidopsis thaliana, Nicotiana benthamiana, and Lycopersicon esculentum (see, e.g., Huang C, Qian Y, Li Z, Zhou X.: Virus-induced gene silencing and its application in plant functional genomics. Sci China Life Sci. 2012; 55 (2):99-108). To confirm whether the T70 gene was responsible for the observed resistance phenotype, VIGS silencing was used to silence T70 in the resistant source S. oleracea. In order to do this, a VIGS construct was made that targeted T70, and this construct was cloned in the K20 vector. Another VIGS construct was made that targeted a different gene (RFP), which was used as a negative control. Table 1 provides the sequences that were used in the VIGS constructs for each gene. The constructs were transformed into spinach using co-cultivation with Agrobacterium tumefaciens strain GV3101, and were used to study the function of T70.

TABLE 1
Target sequences used to produce VIGS constructs

| VIGS | |
|---|---|
| Construct Target | Target Sequence used in VIGS Construct |
| T70 | GGTGAATGTGTTGGCCCTGAAGCTGTATTGGAAACCTTAGAGCCAC |
| | CTTCAAATATCAAGAGCTTATATATATATAATTACAGTGGTACAACA |
| | ATTCCAGTATGGGGAAGAGCAGAGATTAATTGGGCAATCTCCCTCT |
| | CACATCTCGTCGACATCCAGCTTAGTTGTTGTAGTAATTTGCAGGAG |
| | ATGCCAGTGCTGAGTAAACTGCCTCATTTGAAATCGCTGAAACTTGG |
| | ATGGTTGGATAACTTAG (SEQ ID NO: 17) |
| RFP | GAGTTCATGCGCTTCAAGGTGCGCATGGAGGGCTCCGTGAACGGCC |
| | ACAGTTCGAGATCGAGGGCGAGGGCGAGGGCCGCCCCTACGAGGG |
| | CACCCAGACCGCCAAGCTGAAGGTGACCAAGGGCGGCCCCCTGCCC |
| | TTCGCCTGGGACATCCTGTCCCCTCAGTTCCAGTACGGCTCCAAGGC |
| | CTACGTGAAGCACCCCGCCGACATCCCCGACTACTTGAAGCTGTCCT |
| | TCCCCGAGGGCTTCAAGTGGGAGCGCGTGATGAACTTCGAGGACGG |
| | CGGCGTGGTGACCGTGACCCAGGACTCCTCCCTGCAGGACGGCGAG |
| | TTCATCTACAAGGTGAAGCTGCGCGGCACCAACTTCTCCTCCGACGG |
| | CCCCGTAATGCAGAAGAAGACCATGGGCTG (SEQ ID NO: 18) |

### VIGS Silencing Assay and Results

Briefly, S. oleracea lines containing the T70 gene were silenced for T70 using VIGS. Resistant spinach plants were transiently transformed with a T70 silencing construct (generated as described above). Then, the plants were infected with P. farinosa race Pfs 14, which is known to cause downy mildew in spinach. The T70-silenced plants were found to be susceptible to P. farinosa race Pfs14. These results showed that silencing T70 was sufficient to make previously resistant plants susceptible, and demonstrated that T70 was associated with downy mildew resistance.

### Example 3: qPCR Detection of the Expression of Peronospora farinosa Actin in Spinach Infected with Peronospora farinosa

A qPCR experiment was conducted in order to obtain more insight into the response of resistant spinach plants containing the T70 gene to Peronospora farinosa infection. Leaves were harvested from resistant spinach plants, T70-silenced plants, and RFP-silenced plants that had been infected with Peronospora farinosa in the VIGS experiment (described in Example 3). RNA was isolated from these infected leaves, and cDNA was synthesized from the RNA. The expression of Peronospora farinosa actin was analyzed by qPCR using the primers presented in Table 2 (SEQ ID NO: 19, SEQ ID NO: 20, SEQ ID NO: 21, and SEQ ID NO: 22).

TABLE 2
qPCR primers used to determine expression of
Peronospora farinosa and Spinacia oleracea
genes in infected spinach tissues

| Primer name | Sequence |
|---|---|
| P. farinosa | 5′- CTCCCCTCAACCCTAAAGCAA - 3′ |
| actin Fwd | (SEQ ID NO: 19) |
| P. farinosa | 5′- GACAGGACAGCTTGGATGTTTAC - 3′ |
| actin Rv | (SEQ ID NO: 20) |
| S. oleracea | 5′- GCAGGGTCGTTCTTTGAGTC- 3′ |
| elongation | (SEQ ID NO: 21) |
| factor Fwd | |
| S. oleracea | 5′- AGAGGCTCTTCCTGGTGACA - 3′ |
| elongation | (SEQ ID NO: 22) |
| factor Rv | |

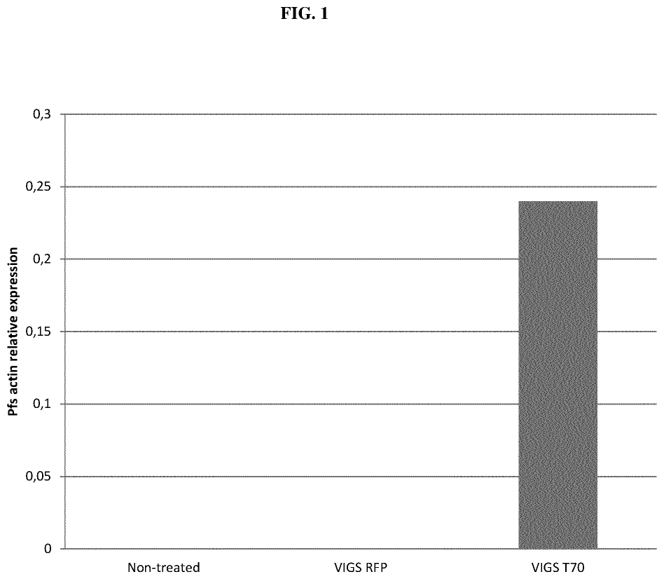

FIG. 1 shows the results of qPCR using primers designed to detect the P. farinosa actin gene (Pfs actin; a housekeeping gene). Three technical replicates were performed, and relative Pfs actin expression was analysed by calculating the relative quantity (RQ=1/(2\hat{ }Cttarget)) and normalised expression (NE=RQtarget/RQref). The relative quantity of the target gene was normalised to expression of the Spinacea oleracea elongation factor gene, which is a housekeeping gene in spinach. Values on the y-axis are relative Pfs actin expression. On the x-axis from left to right: sample obtained from leaves of a plant in which no VIGS silencing construct was used, sample obtained from leaves of a plant in which the RFP VIGS construct was used (negative control), and sample obtained from leaves of a plant in which the T70 VIGS construct was used. Both the samples from the plant in which no VIGS silencing construct was used and the samples from plants in which the RFP VIGS construct was used had a resistant phenotype. Both of these samples expressed the T70 gene. As shown by the qPCR results presented in FIG. 1, Pfs actin was not detectable in these samples using qPCR, and therefore, no P. farinosa was present. In contrast, high transcription levels of the Pfs actin were measured in the sample obtained from T70-silenced plants. Therefore, P. farinosa was present, which correlates with the susceptible phenotype observed in T70-silenced plants.

### Example 4: Downy Mildew Leaf Disc Infection Assay

Spinach plants containing the T70 gene were tested for resistance to different races of the downy mildew pathogen Peronospora farinosa in a test that included the control spinach lines Viroflay, Resistoflay, Califlay, Clermont, Campania, Boeing, and Lazio. Each of these control lines had known resistances and susceptibilities to different Pfs races. The plants used for testing were at least in the second leaf stage, and were not yet flowering.

Resistance was tested using a leaf disc test. Leaves of the different spinach plants were put in trays with moistened paperboard. In order to obtain P. farinosa for infecting the test leaves, seedlings already infected with P. farinosa were suspended in 20 mL water, filtered by cheesecloth, and the flow-through was collected in a spray flask. The tray was spray-inoculated with this Peronospora farinosa suspension. For spray inoculation, leaves were sprayed so that they were completely covered with inoculum, and this complete coverage was checked by making sure that all the discs were wet. The trays were covered with a glass plate and stored in a climate chamber at 15° C. (12 hours light: 12 hours dark cycle). Seven to fourteen days post inoculation, leaves were phenotypically scored by eye for the presence of Peronospora farinosa (Pfs).

The leaves were scored based on symptoms of sporulation on the upper (adaxial side) or lower (abaxial side) side of the leaf disc. The degree of sporulation was qualified by the amount of sporulation and not by the discoloration of the disc. Table 3 provides a detailed description of the disease scoring scale used in the infection assay.

TABLE 3
Leaf disc infection assay scoring scale

| Score | Leaf disc phenotype |
|---|---|
| 9 | No sporulation |
| 7-8 | Small amount of sporulation (maximum of 10 conidiophores) |
| 5-6 | Some sporulation on the edge of the disc |
| 3-4 | Reasonable sporulation in a small area of the disc (25-50% of total |
| | leaf disc area) or a patch of strong sporulation in the edge of the |
| | leaf disc |
| 2 | Reasonable sporulation across 50-75% of leaf disc area |
| 1 | Strong sporulation on 90-100% of leaf disc area |

The infection assay was validated by including control spinach lines with known susceptibilities and resistances to different Pfs races (Viroflay=V, Resistoflay=R, Califlay, Clermont, Campania, Boeing, and Lazio) as well as spinach containing the T70 gene (T70).

Table 4 shows an overview of the leaf disc infection assay results. The assay was performed with isolates of Peronospora farinosa races Pfs1 to Pfs17 on the above-mentioned spinach varieties. The results showed that spinach containing the T70 resistance gene was resistant to at least Peronospora farinosa races Pfs1 to Pfs4, and Pfs7 to Pfs17. For Pfs6 resistance was not determined (ND), but it is expected that the spinach plant will also be resistant to Pfs6. Spinach containing the T70 resistance gene was susceptible to Pfs5. The control lines were shown to each be susceptible to multiple Pfs races. Only the T70 spinach was resistant to the recently identified Pfs17.

TABLE 4
Results of leaf disc infection assay

| Pfs | | | | | | | | |
|---|---|---|---|---|---|---|---|---|
| race | V | R | Califlay | Clermont | Campania | Boeing | Lazio | T70 |
| Pfs1 | 1 | 9 | 9 | 9 | 9 | 9 | 9 | 9 |
| Pfs2 | 1 | 9 | 1 | 9 | 1 | 9 | 9 | 9 |
| Pfs3 | 1 | 1 | 9 | 9 | 9 | 9 | 9 | 9 |
| Pfs4 | 1 | 1 | 1 | 9 | 9 | 9 | 9 | 9 |
| Pfs5 | 1 | 1 | 9 | 1 | 9 | 9 | 9 | 1 |
| Pfs6 | 1 | 1 | 1 | 1 | 1 | 9 | 9 | ND |
| Pfs7 | 1 | 1 | 1 | 1 | 9 | 9 | 9 | 9 |
| Pfs8 | 1 | 1 | 9 | 1 | 1 | 1 | 9 | 9 |
| Pfs9 | 1 | 1 | 9 | 1 | 1 | 9 | 9 | 9 |
| Pfs10 | 1 | 1 | 1 | 1 | 1 | 1 | 9 | 9 |
| Pfs11 | 1 | 1 | 9 | 1 | 9 | 9 | 1 | 9 |
| Pfs12 | 1 | 1 | 9 | 1 | 1 | 1 | 1 | 9 |
| Pfs13 | 1 | 1 | 1 | 1 | 9 | 9 | 1 | 9 |
| Pfs14 | 1 | 1 | 9 | 1 | 1 | 1 | 1 | 9 |
| Pfs15 | 1 | 1 | 1 | 9 | 9 | 9 | 9 | 9 |
| Pfs16 | 1 | 1 | 9 | 1 | 9 | 9 | 1 | 9 |
| Pfs17 | 1 | 1 | 1 | 1 | 1 | 1 | 1 | 9 |

### Example 5: Alignments of Pfs Resistance Gene Coding Sequences and Protein Sequences

The similarity of the novel resistance gene coding sequences (Table 5) and novel resistance proteins (Table 6) was determined using multiple alignment software. The coding sequences of T10 (SEQ ID NO: 1), T70 (SEQ ID NO: 3), T71 (SEQ ID NO: 5), T72 (SEQ ID NO: 7), T75 (SEQ ID NO: 9), T76 (SEQ ID NO: 11), T83 (SEQ ID NO: 13), T89 (SEQ ID NO: 15), T96 (SEQ ID NO: 23) and T253 (SEQ ID NO: 25) were used to generate the results shown in Table 5. The protein sequences of T10 (SEQ ID NO: 2), T70 (SEQ ID NO: 4), T71 (SEQ ID NO: 6), T72 (SEQ ID NO: 8), T75 (SEQ ID NO: 10), T76 (SEQ ID NO: 12), T83 (SEQ ID NO: 14), T89 (SEQ ID NO: 16), and T96 (SEQ ID NO: 24) and T253 (SEQ ID NO: 26) were used to generate the results shown in Table 6. Furthermore, both nucleotide and protein sequences of T18 (SEQ ID No.27, SEQ ID No.28 respectively), T133 (SEQ ID No.29, SEQ ID No.30, respectively), T139 (SEQ ID No.31, SEQ ID No.32, respectively) T170 (SEQ ID No.33, SEQ ID No.34, respectively) and T175 (SEQ ID No.35, SEQ ID No.36, respectively) also have high sequence homology of around 90% or higher among sequences. All resistance genes were highly similar at both the nucleotide and amino acid level. At the amino acid level, T70 had lower similarity to T10, T75, T76, and T83 (<94% identity), but was highly similar to T71, T72, and T89 (>97% identity).

TABLE 5
Percent Identity Matrix of coding sequences of the novel resistance genes

| | T10 | T70 | T71 | T72 | T75 | T76 | T83 | T89 | T96 | T253 |
|---|---|---|---|---|---|---|---|---|---|---|
| T10 | 100.00 | 95.47 | 95.44 | 95.63 | 95.49 | 94.27 | 95.13 | 95.44 | 94.91 | 94.93 |
| T70 | 95.47 | 100.00 | 99.57 | 98.51 | 95.72 | 94.74 | 96.53 | 99.91 | 95.64 | 95.59 |
| T71 | 95.44 | 99.57 | 100.00 | 98.94 | 95.92 | 94.48 | 96.10 | 99.66 | 95.21 | 95.22 |
| T72 | 95.63 | 98.51 | 98.94 | 100.00 | 96.59 | 94.33 | 96.88 | 98.59 | 96.11 | 96.24 |
| T75 | 95.49 | 95.72 | 95.92 | 96.59 | 100.00 | 95.09 | 96.39 | 95.75 | 94.77 | 94.79 |
| T76 | 94.27 | 94.74 | 94.48 | 94.33 | 95.09 | 100.00 | 96.82 | 94.65 | 96.67 | 96.62 |
| T83 | 95.13 | 96.53 | 96.10 | 96.88 | 96.39 | 96.82 | 100.00 | 96.44 | 99.94 | 99.97 |
| T89 | 95.44 | 99.91 | 99.66 | 98.59 | 95.75 | 94.65 | 96.44 | 100.00 | 95.55 | 95.50 |
| T96 | 94.91 | 95.64 | 95.21 | 96.11 | 94.77 | 96.67 | 99.94 | 95.55 | 100.00 | 99.91 |
| T253 | 94.93 | 95.59 | 95.22 | 96.24 | 94.79 | 96.62 | 99.97 | 95.50 | 99.91 | 100.00 |

TABLE 6
Percent Identity Matrix of amino acid sequences of the novel resistance proteins

| | T10 | T70 | T71 | T72 | T75 | T76 | T83 | T89 | T96 | T253 |
|---|---|---|---|---|---|---|---|---|---|---|
| T10 | 100.00 | 92.11 | 91.85 | 92.26 | 92.62 | 90.09 | 91.30 | 91.93 | 90.57 | 90.92 |
| T70 | 92.11 | 100.00 | 99.48 | 97.50 | 93.23 | 91.41 | 93.84 | 99.83 | 92.44 | 92.79 |
| T71 | 91.85 | 99.48 | 100.00 | 98.02 | 93.32 | 91.23 | 93.32 | 99.66 | 91.92 | 92.27 |
| T72 | 92.26 | 97.50 | 98.02 | 100.00 | 94.36 | 90.97 | 94.75 | 97.67 | 93.45 | 94.03 |
| T75 | 92.62 | 93.23 | 93.32 | 94.36 | 100.00 | 92.36 | 94.01 | 93.23 | 91.67 | 92.01 |
| T76 | 90.09 | 91.41 | 91.23 | 90.97 | 92.36 | 100.00 | 94.79 | 91.23 | 94.19 | 94.55 |
| T83 | 91.30 | 93.84 | 93.32 | 94.75 | 94.01 | 94.79 | 100.00 | 93.66 | 99.48 | 100.00 |
| T89 | 91.93 | 99.83 | 99.66 | 97.67 | 93.23 | 91.23 | 93.66 | 100.00 | 92.27 | 92.62 |
| T96 | 90.57 | 92.44 | 91.92 | 93.45 | 91.67 | 94.19 | 99.48 | 92.27 | 100.00 | 99.48 |
| T253 | 90.92 | 92.79 | 92.27 | 94.03 | 92.01 | 94.55 | 100.00 | 92.62 | 99.48 | 100.00 |

### DEPOSIT INFORMATION

A deposit of spinach (Spinacia oleracea 2017.02544-B/SNNLENBL 19011503) is maintained by Enza Zaden USA, Inc., having an address at 7 Harris Place, Salinas, Calif. 93901, United States. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to a deposit of at least 2,500 seeds of the same variety with the National Collection of Industrial, Food and Marine Bacteria Ltd. (NCIMB Ltd), Ferguson Building, Craibstone Estate, Bucksburn, Aberdeen, AB21 9YA, United Kingdom.

At least 2500 seeds of spinach (Spinacia oleracea 2017.02544-B/SNNLENBL 19011503) were deposited on Feb. 21, 2019 according to the Budapest Treaty in the National Collection of Industrial, Food and Marine Bacteria Ltd (NCIMB Ltd), Ferguson Building, Craibstone Estate, Bucksburn, Aberdeen, AB21 9YA, United Kingdom. The deposit has been assigned NCIMB number 43360. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed.

The deposit will be maintained in the NCIMB depository, which is a public depository, for a period of at least 30 years, or at least 5 years after the most recent request for a sample of the deposit, or for the effective life of the patent, whichever is longer, and will be replaced if a deposit becomes nonviable during that period.

1. A spinach plant that is resistant to downy mildew caused by Peronospora farinosa (Pfs), wherein the spinach plant comprises one or more resistance genes, wherein said one or more resistance genes encode for a protein having at least 85% sequence identity with SEQ ID No. 4, wherein the protein comprises a conserved amino acid sequence KDHKIEKE and a conserved amino acid sequence LSNNRNLKIL.

2. Spinach plant according to claim 1, wherein said one or more resistance genes encode for a protein, wherein said a protein is selected from the group consisting of SEQ ID No.2, SEQ ID No.4, SEQ ID No.6, SEQ ID No.8, SEQ ID No.14, SEQ ID No.16, SEQ ID No.24, SEQ ID No.26, SEQ ID No.28, SEQ ID No.30, SEQ ID No.32, SEQ ID No.34 and SEQ ID No.36.

3. Spinach plant according to claim 1 or 2, wherein said one or more resistance genes comprise a coding sequence selected from the group consisting of SEQ ID No.1, SEQ ID No.3, SEQ ID No.5, SEQ ID No.7, SEQ ID No.13, SEQ ID No.15, SEQ ID No.23, SEQ ID No.25, SEQ ID No.27, SEQ ID No.29, SEQ ID No.31, SEQ ID No.33 and SEQ ID No.35.

4. Spinach plant according to any one of the claims 1 to 3, wherein said spinach plant comprises the one or more resistance genes selected from the group consisting of SEQ ID No.3, SEQ ID No.5, SEQ ID No.7, and SEQ ID No.15.

5. A spinach plant that is resistant to downy mildew caused by Peronospora farinosa (Pfs), wherein the spinach plant comprises one or more resistance genes comprise a coding sequence selected from the group consisting of SEQ ID No.1, SEQ ID No.3, SEQ ID No.5, SEQ ID No.7, SEQ ID No.9, SEQ ID No.11, SEQ ID No.13, and SEQ ID No.15, SEQ ID No.23, SEQ ID No.25, SEQ ID No.27, SEQ ID No.29, SEQ ID No.31, SEQ ID No.33 and SEQ ID No.35.

6. Spinach plant according to claim 5, wherein said one or more resistance genes encode for a protein selected from the group consisting of SEQ ID No.2, SEQ ID No.4, SEQ ID No.6, SEQ ID No.8, SEQ ID 10, SEQ ID No.12, SEQ ID No.14, and SEQ ID No.16, SEQ ID No.24, SEQ ID No.26, SEQ ID No.28, SEQ ID No.30, SEQ ID No.32, SEQ ID No.34 and SEQ ID No.36.

7. Spinach plant according to any one of the claims 1 to 6, wherein said plant is at least resistant to Peronospora farinosa races Pfs1 to Pfs4, and Pfs7 to Pfs17.

8. Spinach plant according to any one of the claims 1 to 7, wherein said one or more resistance genes is derived from deposit number NCIMB 43360.

9. Seed obtained from a spinach plant according to any one of the claims 1 to 8, the seed comprising one or more resistance genes, wherein said one or more resistance genes encode for a protein having at least 85% sequence identity with SEQ ID No. 4, wherein the protein comprises a conserved amino acid sequence KDHKIEKE and a conserved amino acid sequence LSNNRNLKIL.

10. A resistance gene that confers resistance to downy mildew in spinach plants, wherein the gene encodes for a protein that has at least 85% sequence identity with SEQ ID No. 4.

11. Resistance gene according to claim 10, wherein the gene comprises a coding sequence selected from the group consisting of SEQ ID No.1, SEQ ID No.3, SEQ ID No.5, SEQ ID No.7, SEQ ID No.13, SEQ ID No.15, SEQ ID No.23, SEQ ID No.25, SEQ ID No.27, SEQ ID No.29, SEQ ID No.31, SEQ ID No.33 and SEQ ID No.35.

12. Resistance gene according to any one of the claim 10 or 11, wherein said resistance gene encodes for a protein, wherein said protein is comprised of a conserved amino acid sequence KDHKIEKE and a conserved amino acid sequence LSNNRNLKIL.

13. Resistance gene according to any one of the claims 10 to 12, wherein the coding sequence of said resistance gene is selected form the group consisting of SEQ ID No.3, SEQ ID No.5, SEQ ID No.7, and SEQ ID No.15.

14. Resistance gene according to any one of the claims 10 to 13, wherein the gene provides resistance to Peronospora farinosa races Pfs1 to Pfs4, and Pfs7 to Pfs17 in spinach.

15. Method for providing a spinach plant that is resistant to downy mildew, wherein the method comprises the steps of introducing one or more resistance genes into the genome of the spinach plant, wherein the one or more resistance genes are selected from the group consisting of SEQ ID No.1, SEQ ID No.3, SEQ ID No.5, SEQ ID No.7, SEQ ID No.13, SEQ ID No.15, SEQ ID No.23, SEQ ID No.25, SEQ ID No.27, SEQ ID No.29, SEQ ID No.31, SEQ ID No.33 and SEQ ID No.35.

16. Method according to claim 15, wherein the introduction of the one or more resistance genes is achieved by genome editing techniques, CRISPR Cas, or mutagenisis techniques.

17. Method for providing a spinach plant that is resistant to downy mildew, wherein the method comprises the steps of
a) providing a spinach plant comprising one or more resistance gene(s), wherein said resistance gene(s) is according to any one of the claims 10 to 14,
b) crossing the spinach plant of step a) with a susceptible spinach plant,
c) optionally, selfing the plant obtained in step b) for at least one time,
d) selecting the plants that are resistant to downy mildew.

18. Method according to claim 17, wherein the coding sequence of said one or more resistance genes is selected from the group consisting of SEQ ID No.3, SEQ ID No.5, SEQ ID No.7, and SEQ ID No.15.

19. Method according to any one of claims 15 to 18, wherein the spinach plant is resistant to downy mildew caused by Peronospora farinosa races Pfs1 to Pfs4, and Pfs7 to Pfs 17.

20. Method according to any one of claims 17 to 19, wherein the one or more resistance gene(s) is obtained from deposit number NCIMB 43360.
